SEQUENCE LISTING (30 sequences)

SEQ ID NO: 1 - protein, 222 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 2 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 456 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKLPETGG

SEQ ID NO: 5 - protein, 214 aa, synthetic construct; identical to SEQ ID NO: 3

SEQ ID NO: 6 - protein, 458 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGALPETGG

SEQ ID NO: 7 - protein, 220 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECLPETGG

SEQ ID NO: 8 - protein, 450 aa, synthetic construct; identical to SEQ ID NO: 2

SEQ ID NO: 9 - protein, 220 aa, synthetic construct; identical to SEQ ID NO: 7

SEQ ID NO: 10 - protein, 456 aa, synthetic construct; identical to SEQ ID NO: 4

SEQ ID NO: 11 - protein, 220 aa, synthetic construct; identical to SEQ ID NO: 7

SEQ ID NO: 12 - protein, 458 aa, synthetic construct; identical to SEQ ID NO: 6

SEQ ID NO: 13 - protein, 222 aa, synthetic construct; identical to SEQ ID NO: 1

SEQ ID NO: 14 - protein, 456 aa, synthetic construct; identical to SEQ ID NO: 4

SEQ ID NO: 15 - protein, 222 aa, synthetic construct; identical to SEQ ID NO: 1

SEQ ID NO: 16 - protein, 458 aa, synthetic construct; identical to SEQ ID NO: 6

SEQ ID NO: 17 - protein, 11 aa, synthetic construct
RASQDVNTAVA

SEQ ID NO: 18 - protein, 7 aa, synthetic construct
SASFLYS

SEQ ID NO: 19 - protein, 9 aa, synthetic construct
QQHYTTPPT

SEQ ID NO: 20 - protein, 5 aa, synthetic construct
DTYIH

SEQ ID NO: 21 - protein, 17 aa, synthetic construct
RIYPTNGYTRYADSVKG

SEQ ID NO: 22 - protein, 11 aa, synthetic construct
WGGDGFYAMDY

SEQ ID NO: 23 - protein, 110 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 24 - protein, 120 aa, synthetic construct [sequence omitted]

SEQ ID NO: 25 - protein, 5 aa, synthetic construct
GGGGS

SEQ ID NO: 26 - protein, 10 aa, synthetic construct
GGGGSGGGGS

SEQ ID NO: 27 - protein, 15 aa, synthetic construct
GGGGSGGGGSGGGGS

SEQ ID NO: 28 - protein, 6 aa, synthetic construct
LPETGG

SEQ ID NO: 29 - protein, 8 aa, synthetic construct
GALPETGG

SEQ ID NO: 30 - protein, 5 aa, synthetic construct
  source 1..5: note = X=Gn, n represents the number of G.
LPETX

(19) United States
(12) Patent Application Publication
(10) Pub. No.: US 2025/0312470 A1
(43) Pub. Date: Oct. 9, 2025

(54) A PHARMACEUTICAL COMPOSITION OF ANTI-HER2 ANTIBODY-IMMUNE AGONIST CONJUGATE AND APPLICATIONS THEREOF

(71) Applicants: GENEQUANTUM HEALTHCARE (SUZHOU) CO., LTD., Suzhou (CN); GENEQUANTUM MEDICINE (SUZHOU) CO., LTD., Suzhou (CN)

(72) Inventors: Xuesong LI, Suzhou (CN); Paul H. SONG, Suzhou (CN); Gang QIN, Suzhou (CN)

(21) Appl. No.: 19/109,203

(22) PCT Filed: Sep. 6, 2023
(86) PCT No.: PCT/CN2023/117282

§ 371 Date: Mar. 6, 2025

(30) Foreign Application Priority Data
Sep. 6, 2022 (WO) PCT/CN2022/117192

(51) Int. Cl. A61K 47/68; A61K 47/22; A61K 47/26; C07K 16/32

(52) U.S. Cl. CPC A61K 47/6803; A61K 47/22; A61K 47/26; A61K 47/6855; A61K 47/6889; C07K 16/32

(57) ABSTRACT

Provided herein is a pharmaceutical composition of an antibody-immune agonist conjugate (AIAC), as a novel type of tumor targeting therapy.

### CROSS REFERENCE TO RELATED APPLICATIONS

This application is a National Stage Entry under 35 U.S.C. § 371 of International Application No. PCT/CN2023/117282 filed Sep. 6, 2023, which claims the benefit and priority of International Application No. PCT/CN2022/117192 filed Sep. 6, 2022, the contents of each of which are incorporated herein by reference.

### REFERENCE TO A SEQUENCE LISTING

This application contains a Sequence Listing as a separate part of the disclosure. The contents of the Sequence Listing (2025-03-06-GQH-25-US-Sequence Listing.xml; Size: 32,748 bytes; and Date of Creation: Mar. 5, 2025) are incorporated herein by reference.

### TECHNICAL FIELD

The present disclosure relates to the biopharmaceutical field, particularly to a pharmaceutical composition of an antibody-immune agonist conjugate (AIAC), as a novel type of tumor targeting therapy.

### BACKGROUND

In the developmental and clinical circumstances, targeted delivery of therapeutic agents remains a great challenge for cancer therapies. The present targeting molecule-drug conjugates that are approved by FDA are mainly antibody-drug conjugates (ADCs) wherein the drug (warhead) is usually a small molecule cytotoxin.

Human epidermal growth factor receptor 2 (HER2) is a member of epidermal growth factor receptor family having tyrosine kinase activity. Amplification or overexpression of HER2 occurs in approximately 15-30% of breast cancers and 10-30% of gastric/gastroesophageal cancers. HER2 overexpression has also been seen in other cancers like ovary, endometrium, bladder, lung, colon, and head and neck (Iqbal N. et al, Mol Biol Int. 2014:852748). Although the efficacy of HER2-targeted therapies, such as HER2 directed antibody or antibody-drug conjugate (ADC), substantially improves the life expectancy of patients with HER2-positive disease, by nature, HER2-positive breast cancer is still a more aggressive form of the disease, with a poorer prognosis and worse outcomes than for patients with HER2-negative (and HR-positive) disease. Moreover, the therapeutic results have been proved disappointing in other HER2 overexpressing cancers. One of the numerous reasons for the poor outcome is that patents administered with HER2-targeting therapy become resistant. Immune escape of the tumor cells incurs to the process.

Immunotherapy is a new modality of cancer therapy that has shown great power. While immune checkpoint inhibitors represented by CLTA-4 and PD-1/L1 monoclonal antibody, which are basically T cell-based therapy, was approved for various cancer indications, there are also a lot of efforts exploring other mechanisms of immune system to fight against cancers. Targeting myeloid cells, majorly macrophages, DCs, has emerged as a promising direction. Activating macrophages and DCs by agonists or by macrophage checkpoint inhibitors enhances not only their capacity of phagocytosis to clear tumor cells, but also their functions of antigen presentation, which would more robustly ignite adaptive anti-tumor immunity.

TLR7/8 are two important pattern recognition receptors that are located in the endosomal membrane of macrophages, DCs, and monocytes. They naturally sense the ssRNAs derived from virus, mediate the activation of immune cells and release of pro-inflammation cytokines. A lot of researches have demonstrated that TLR7/8 agonists have anti-tumor activity. Imiquimod, a TLR7 agonist, has been approved for the treatment of genital warts, superficial basal cell carcinoma, and actinic keratosis by topical administration. Resiquimod, a TLR7/8 dual agonist, has been approved for the treatment of cutaneous T cell lymphoma. Nevertheless, the side effects induced by systemic administration of TLR7/8 agonist restricted their usage in broader spectrum of cancers.

The present disclosure provides a pharmaceutical composition of an HER2 directed antibody-immune agonist conjugate (AIAC), a novel drug of tumor targeting immunotherapy.

As known in the art, active pharmaceutical ingredients (APIs) determine the use of the drugs, and the prescriptions of pharmaceutical formulation are closely related to the nature of APIs. For example, pH of the formulations can affect the stability of the APIs, and excipients not only have an impact on the solubility and dissolution of APIs, but also have a significant impact on the properties of APIs such as permeability and absorption. Thus, it is very necessary to develop formulations for specific APIs.

### SUMMARY

In one aspect, provided is an antibody-drug conjugate of formula (II-1) and/or (II-2):

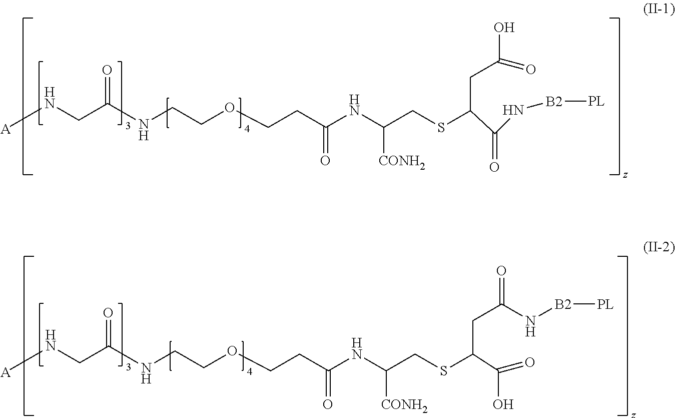

wherein B2 is —(CH₂)ₖ(CO)—NH—(C₂H₄—O)ⱼ— or —(CH₂)ₖC(O)—(NH—CR¹R²—C(O))_{d}—, k is an integer of 1 to 5, j is an integer of 1 to 3, d is an integer of 1 or 2, R¹ and R² are each independently selected from hydrogen, —OH, —NH₂, —C₁₋₆ alkyl;
PL is a payload which is linked to the B2 moiety,
preferably, PL is Resiquimod

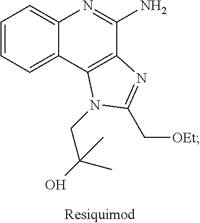 [SMILES: CCOCc1nc2c(N)nc3ccccc3c2n1CC(C)(C)O]

z is an integer of 1 to 4; preferably 1 to 2;

A is an antibody comprises a V_{L} and a V_{H}, wherein the V_{L} comprises LCDR1 having the amino acid sequence of SEQ ID NO: 17, LCDR2 having the amino acid sequence of SEQ ID NO: 18, and LCDR3 having the amino acid sequence of SEQ ID NO: 19, wherein the V_{H} comprises HCDR1 having the amino acid sequence of SEQ ID NO: 20, HCDR2 having the amino acid sequence of SEQ ID NO: 21, and HCDR3 having the amino acid sequence of SEQ ID NO: 22; wherein the antibody is modified by introduction of the ligase donor substrate recognition sequence. In a preferred embodiment, the antibody comprises a V_{L} having the amino acid sequence of SEQ ID NO: 23 and a V_{H} having the amino acid sequence of SEQ ID NO: 24.

In one aspect, provided is an antibody-drug conjugate of formula (II-1) and/or (II-2):

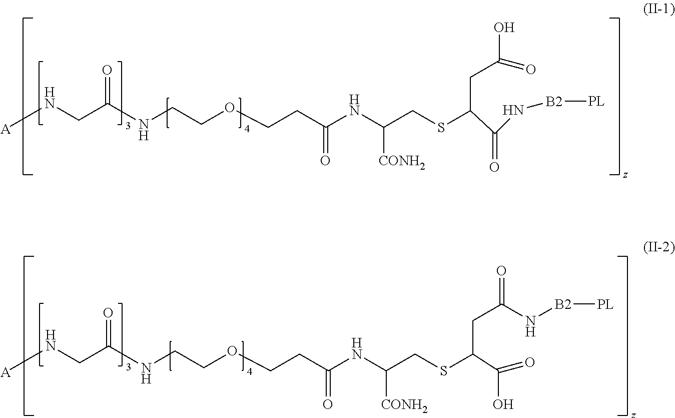

wherein B2 is —(CH₂)ₖ(CO)—NH—(C₂H₄—O)ⱼ— or —(CH₂)ₖC(O)—(NH—CR¹R²—C(O))_{d}—, k is an integer of 1 to 5, j is an integer of 1 to 3, d is an integer of 1 or 2, R¹ and R² are each independently selected from hydrogen, —OH, —NH₂, —C₁₋₆ alkyl;
PL is a payload which is linked to the B2 moiety,
preferably, PL is Resiquimod

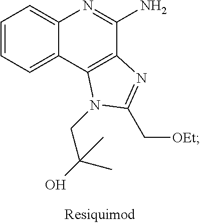 [SMILES: CCOCc1nc2c(N)nc3ccccc3c2n1CC(C)(C)O]

z is an integer of 1 to 4; preferably 1 to 2;
A is an antibody comprising a light chain having the amino acid sequence of SEQ ID NO: 1 and a heavy chain having the amino acid sequence of SEQ ID NO: 2; or
a light chain having the amino acid sequence of SEQ ID NO: 3 and a heavy chain having the amino acid sequence of SEQ ID NO: 4; or
a light chain having the amino acid sequence of SEQ ID NO: 5 and a heavy chain having the amino acid sequence of SEQ ID NO: 6; or
a light chain having the amino acid sequence of SEQ ID NO: 7 and a heavy chain having the amino acid sequence of SEQ ID NO: 8; or
a light chain having the amino acid sequence of SEQ ID NO: 9 and a heavy chain having the amino acid sequence of SEQ ID NO: 10; or
a light chain having the amino acid sequence of SEQ ID NO: 11 and a heavy chain having the amino acid sequence of SEQ ID NO: 12; or
a light chain having the amino acid sequence of SEQ ID NO: 13 and a heavy chain having the amino acid sequence of SEQ ID NO: 14; or
a light chain having the amino acid sequence of SEQ ID NO: 15 and a heavy chain having the amino acid sequence of SEQ ID NO: 16.

The antibody-immune agonist conjugates (AIACs) of the present disclosure provide a novel type of tumor targeting therapy. In vitro experiments demonstrate that the AIACs can induce higher TNF production compared to naked unmodified antibody. In vivo experiments of the AIACs show anti-tumor effect.

In another aspect, provided is a pharmaceutical composition comprising a prophylactically or therapeutically effective amount of a conjugate of formula (II-1) and/or (II-2), and at least one pharmaceutically acceptable carrier.

### BRIEF DESCRIPTION OF THE DRAWINGS

FIG. 1: TNFα induction activity in human PBMC and NCI N87 co-culture assay for conjugate AC102-6-1-1 and the corresponding naked unmodified antibody Ab0001 (Trastuzumab), and agonist Resiquimod.

FIG. 2: TNFα induction activity in human PBMC and NCI N87 co-culture assay for conjugates AC102-6-1-1, AC102-8-1-1 and their corresponding naked unmodified antibody Ab0001.

FIG. 3: TNFα induction activity of AC102-6-1-1 and antibody in co-culture of PBMC with either NCI N87 or MDA-MB-468 cells.

FIG. 4: TNFα induction activity of AC102-8-1-1 and antibody in co-culture of PBMC with HCC1954 cells with high expression of HER2.

FIG. 5: TNFα induction activity of AC102-8-1-1 and antibody in co-culture of PBMC with SK-BR-3 cells with high expression of HER2.

FIG. 6: TNFα induction activity of AC102-8-1-1 and antibody in co-culture of PBMC with BT474 cells with high expression of HER2.

FIG. 7: TNFα induction activity of AC102-8-1-1 and antibody in co-culture of PBMC with JIMT1 cells with medium expression of HER2.

FIG. 8: TNFα induction activity of AC102-8-1-1 and antibody in co-culture of PBMC with Colo205 cells with low expression of HER2.

FIG. 9: TNFα induction activity of AC102-8-1-1 and antibody in co-culture of PBMC with MDA-MB-468 cells with low expression of HER2.

FIG. 10: INF-y induction activity of AC102-6-1-1 and antibody in co-culture of PBMC with SK-BR-3 cells.

FIG. 11: INF-y induction activity of AC102-6-1-1 and antibody in co-culture of PBMC with HCC1954 cells.

FIG. 12: Tumor volume change over time in SCID Beige mice with NCI N87 CDX model dosed with: Vehicle (PBS pH 6.5), antibody, and conjugates AC102-6-1-1 and AC102-8-1-1 at 5 mg/kg.

FIG. 13: Tumor volume change over time in SCID Beige mice with NCI N87 CDX model dosed with 0.5, 1, and 3 mg/kg AC102-8-1-1.

FIG. 14: Tumor volume change over time in SCID Beige mice with JIMT1 CDX model dosed with 5 mg/kg AC102-8-1-1.

FIG. 15: Tumor volume change over time in MC38 model overexpressing hHER2 dosed with 3 mg/kg and 10 mg/kg AC102-6-1-1.

FIG. 16: Tumor volume change over time in MC38 model overexpressing hHER2 dosed with 3 mg/kg and 10 mg/kg AC102-8-1-1.

### DETAILED DESCRIPTION

The specific embodiments are provided below to illustrate technical contents of the present disclosure. Those skilled in the art can easily understand other advantages and effects of the present disclosure through the contents disclosed in the specification. The present disclosure can also be implemented or applied through other different specific embodiments. Various modifications and variations can be made by those skilled in the art without departing from the spirit of the present disclosure.

### Definitions

Unless otherwise defined hereinafter, all technical and scientific terms used herein have the same meaning as commonly understood by those skilled in the art. The techniques used herein refer to those that are generally understood in the art, including the variants and equivalent substitutions that are obvious to those skilled in the art. While the following terms are believed to be readily comprehensible by those skilled in the art, the following definitions are set forth to better illustrate the present disclosure. When a trade name is present herein, it refers to the corresponding commodity or the active ingredient thereof. All patents, published patents applications and publications cited herein are hereby incorporated by reference.

When a certain amount, concentration, or other value or parameter is set forth in the form of a range, a preferred range, or a preferred upper limit or a preferred lower limit, it should be understood that it is equivalent to specifically revealing any range formed by combining any upper limit or preferred value with any lower limit or preferred value, regardless of whether the said range is explicitly recited. Unless otherwise stated, the numerical ranges listed herein are intended to include the endpoints of the range and all integers and fractions (decimals) within the range. For example, the expression “i is an integer of 2 to 20” means that i is any integer of 2 to 20, for example, i can be 2, 3, 4, 5, 6, 7, 8, 9, 10, 11, 12, 13, 14, 15, 16, 17, 18, 19 or 20. Other similar expressions should also be understood in a similar manner.

Unless otherwise stated herein, singular forms like “a” and “the” include the plural forms. The expression “one or more” or “at least one” may mean 1, 2, 3, 4, 5, 6, 7, 8, 9 or more.

The terms “about” and “approximately”, when used in connection with a numerical variable, generally mean that the value of the variable and all values of the variable are within experimental error (for example, within a 95% confidence interval for the mean) or within ±10% of a specified value, or a wider range.

The expression “comprising” or similar expressions “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

As used herein, the concentration “%” represents a mass volume concentration in the unit of g/ml. For example, 9% sucrose solution represents that 9 g of sucrose is dissolved in the solvent to form 100 ml of solution, which means that the solution contains 9 g sucrose per 100 ml.

The amount of buffer in the present disclosure refers to the total amount of the buffer pair in the buffer system constituting the buffer. In some embodiments, molar concentration is used as the unit of the amount of the buffer, and its numerical value refers to the molar concentration of the buffer pair in the buffer system of the buffer. For example, when a histidine buffer consisting of L-Histidine and L-histidine hydrochloride is used, the given concentration (e.g., 10 mM) of the histidine buffer is a combined concentration of L-Histidine and L-histidine hydrochloride (e.g., L-Histidine is 5 mM, and L-histidine hydrochloride is 5 mM; or L-Histidine is 6 mM, and L-histidine hydrochloride is 4 mM; or L-Histidine is 3.46 mM, and L-histidine hydrochloride is 6.54 mM, etc.).

In the solid state, trehalose is usually present in the form of trehalose dihydrate. In some embodiments, trehalose dihydrate can be used for preparation, and corresponding amounts of other forms of trehalose can also be used for preparation, and obtained formulations contain the same concentration of trehalose. When describing the content of trehalose in the formulation using trehalose dihydrate in this present disclosure, the formulations contain the stated amount of trehalose dihydrate or corresponding amounts of trehalose or other forms of trehalose or their combinations, and vice versa.

The term “targeting molecule” refers to a molecule that has an affinity for a particular target (e.g., receptor, cell surface protein, cytokine, etc.). A targeting molecule can deliver the payload to a specific site in vivo through targeted delivery. A targeting molecule can recognize one or more targets. The specific target site is defined by the targets it recognizes. For example, a targeting molecule that targets a receptor can deliver a payload to a site containing a large number of the receptors. Examples of targeting molecules include, but are not limited to antibodies, antibody fragments, binding proteins for a given antigen, antibody mimics, scaffold proteins having affinity for a given target, ligands, and the like.

As used herein, the term “antibody” is used in a broad way and particularly includes an intact monoclonal antibody, a polyclonal antibody, a monospecific antibody, a multispecific antibody (e.g., a bispecific antibody), and an antibody fragment, as long as they have the desired biological activity. The antibody may be of any subtype (such as IgG, IgE, IgM, IgD, and IgA) or subclass, and may be derived from any suitable species. In some embodiments, the antibody is of human or murine origin. The antibody may also be a fully human antibody, humanized antibody or chimeric antibody prepared by recombinant methods.

Monoclonal antibodies are used herein to refer to antibodies obtained from a substantially homogeneous population of antibodies, i.e., the individual antibodies constituting the population are identical except for a small number of possible natural mutations. Monoclonal antibodies are highly specific for a single antigenic site, multiple antigenic sites or different epitopes of the same antigen. The word “monoclonal” refers to that the characteristics of the antibody are derived from a substantially homogeneous population of antibodies and are not to be construed as requiring some particular methods to produce the antibody.

An intact antibody or full-length antibody essentially comprises the antigen-binding variable region(s) as well as the light chain constant region(s) (C_{L}) and heavy chain constant region(s) (C_{H}), which could include C_{H}1, C_{H}2, C_{H}3 and/or C_{H}4, depending on the subtype of the antibody. An antigen-binding variable region (also known as a fragment variable region, Fv fragment) typically comprises a light chain variable region (V_{L}) and a heavy chain variable region (V_{H}). A constant region can be a constant region with a native sequence (such as a constant region with human native sequences) or an amino acid sequence variant thereof. The variable region recognizes and interacts with the target antigen. The constant region can be recognized by and interacts with the immune system.

As used herein, the term “heavy chain constant region (CH)” includes amino acid sequences derived from an intact antibody or full-length antibody heavy chain. A polypeptide comprising a heavy chain constant region comprises at least one of: a C_{H}1 domain, a hinge (e.g., upper, middle, and/or lower hinge region) domain, a C_{H}2 domain, a C_{H}3 domain, a C_{H}4 domain, or a variant or fragment thereof. For example, an antigen-binding polypeptide for use in the disclosure may comprise a polypeptide chain comprising a C_{H}1 domain; a polypeptide chain comprising a C_{H}1 domain, at least a portion of a hinge domain, and a C_{H}2 domain; a polypeptide chain comprising a C_{H}1 domain and a C_{H}3 domain; a polypeptide chain comprising a C_{H}1 domain, at least a portion of a hinge domain, and a C_{H}3 domain, or a polypeptide chain comprising a C_{H}1 domain, at least a portion of a hinge domain, a C_{H}2 domain, and a C_{H}3 domain.

As used herein, “C.” refers to a constant region of a light chain. The subunit structures and three dimensional configuration of the constant regions of the various antibody classes are well known. As used herein, the term “V_{H} domain” includes the amino terminal variable domain of an antibody heavy chain and the term “C_{H}1 domain” includes the first (most amino terminal) constant region domain of an antibody heavy chain. The C_{H}1 domain is adjacent to the V_{H} domain and is amino terminal to the hinge region of an antibody heavy chain molecule.

As used herein, “V_{L}” refers to a variable region of a light chain.

An antibody fragment may comprise a portion of an intact antibody, preferably its antigen binding region or variable region. Examples of antibody fragments include Fab, Fab′, F(ab′)₂, Fd fragment consisting of V_{H} and C_{H}1 domains, Fv fragment, single-domain antibody (dAb) fragment, and isolated complementarity determining region (CDR). The Fab fragment is an antibody fragment obtained by papain digestion of a full-length immunoglobulin, or a fragment having the same structure produced by, for example, recombinant expression. A Fab fragment comprises a light chain (comprising a V_{L} and a C_{L}) and another chain, wherein the said other chain comprises a variable domain of the heavy chain (V_{H}) and a constant region domain of the heavy chain (C_{H}1). The F(ab′)₂ fragment is an antibody fragment obtained by pepsin digestion of an immunoglobulin at pH 4.0-4.5, or a fragment having the same structure produced by, for example, recombinant expression. The F(ab′)₂ fragment essentially comprises two Fab fragments, wherein each heavy chain portion comprises a few additional amino acids, including the cysteines that form disulfide bonds connecting the two fragments. A Fab′ fragment is a fragment comprising one half of a F(ab′)₂ fragment (one heavy chain and one light chain). The antibody fragment may comprise a plurality of chains joined together, for example, via a disulfide bond and/or via a peptide linking unit. Examples of antibody fragments also include single-chain Fv (scFv), Fv, dsFv, diabody, Fd and Fd′ fragments, and other fragments, including modified fragments. An antibody fragment typically comprises at least or about 50 amino acids, and typically at east or about 200 amino acids. An antigen-binding fragment can include any antibody fragment that, when inserted into an antibody framework (e.g., by substitution of the corresponding region), can result in an antibody that immunospecifically binds to the antigen.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The exact residue numbers which encompass a particular CDR will vary depending on the sequence and size of the CDR. Those skilled in the art can routinely determine which residues comprise a particular CDR given the variable region amino acid sequence of the antibody.

As used herein, “HCDR” refers to a complementarity determining region of a heavy chain.

As used herein, “LCDR” refers to a complementarity determining region of a light chain.

Antibodies according to the present disclosure can be prepared using techniques well known in the art, such as the following techniques or a combination thereof: recombinant techniques, phage display techniques, synthetic techniques, or other techniques known in the art. For example, a genetically engineered recombinant antibody (or antibody mimic) can be expressed by a suitable culture system (e.g., E. coli or mammalian cells). The engineering of antibody can refer to, for example, the introduction of a ligase-specific recognition sequence at its terminals.

HER2 refers to human epidermal growth factor receptor-2, which belongs to the epidermal growth factor (EGFR) receptor tyrosine kinase family. In the present disclosure, the terms ErbB2 and HER2 have the same meaning and can be used interchangeably.

As used herein, the term “targeting molecule-drug conjugate” is referred to as “conjugate”. Examples of conjugates include, but are not limited to, antibody-drug conjugates.

As used herein, the expression “the concentration of AIAC” has the same meaning as “the concentration of the protein of AIAC”, and they can be used interchangeably.

A small molecule compound refers to a molecule with a size comparable to that of an organic molecule commonly used in medicine. The term does not encompass biological macromolecules (e.g., proteins, nucleic acids, etc.), but encompasses low molecular weight peptides or derivatives thereof, such as dipeptides, tripeptides, tetrapeptides, pentapeptides, and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da.

Immune agonist refers to an agonist which can induce or enhance immune response to the tumor, such through activation of immune cells, including but not limited to DCs, B cells, macrophages, NK cells, and T cells. The non-limiting examples of immune agonists such as TLR agonists, including but not limited to agonists of TLR7 and/or TLR8 and/or TLR9 (e.g., Imiquimod, Resiquimod, 852A and VTX-2337) and STING agonists (e.g., ADU-S100 and MK-1454) are known in the art.

Linking unit refers to a functional group that covalently bonds two or more moieties in a compound or material. For example, the linking unit can serve to covalently bond adjuvant moieties of targeting molecule(s) and/or payload(s).

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid such as Leu, Gln, etc., a sequence containing a plurality of amino acids, for example, a sequence containing two amino acids such as GA, etc., or, for example, GGGGS, GGGGSGGGGS, GGGGSGGGGSGGGGS, etc. Other examples of spacers include, for example, self-immolative spacers such as PABC (p-benzyloxycarbonyl), and the like.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical has two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆—), butylene (—C₄H₈—), pentylene (—C₅H₁₀—), hexylene (—C₆H₁₂—), 1-methylethylene (—CH(CH₃)CH₂—), 2-methylethylene (—CH₂CH(CH₃)—), methylpropylene, ethylpropylene, and the like.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond. For example, when describing a combination of a C₁₋₄ alkylene with one of the groups including —CH₂—, —NH—, —(CO)—, —NH(CO)—, —(CO)NH—, the C₁₋₄ alkylene may form a linear connection with the above groups, such as C₁₋₄ alkylene-CH₂—, C₁₋₄ alkylene-NH—, C₁₋₄ alkylene-(CO)—, C₁₋₄ alkylene-NH(CO)—, C₁₋₄ alkylene-(CO)NH—, —CH₂—C₁₋₄ alkylene, —NH—C₁₋₄ alkylene, —(CO)—C₁₋₄ alkylene, —NH(CO)—C₁₋₄ alkylene, —(CO)NH—C₁₋₄ alkylene. The resulting bivalent structure can be further connected to other moieties of the molecule.

The term “acidic buffer” refers to a buffer with pH<7, for example, the buffer with pH=4.0-6.0, pH=4.0, pH=4.5, pH=5.0, pH=5.5 or PH=6.0.

The “stabilizer” refers to a chemical that can increase the stability of solutions, colloids, solids, mixtures, etc., and has the functions of slowing down the reaction, maintaining chemical balance, reducing surface tension, and preventing light, thermal decomposition or oxidative decomposition, for example, the stabilizer may be sucrose, trehalose dihydrate, sorbitol, and the combinations thereof.

As used herein, “surfactant” refers to a substance that can obviously change the interface state of the solution system by adding a small amount. Examples of surfactant include, but not limited to, tween 20 (PS 20), tween 80 (PS 80), span 20 (SP 20), span 80 (SP 80). In some embodiment, surfactant can be used as stabilizer.

As used herein, “Tm” refers to melting temperature indicating the temperature at which protein begins to denature.

As used herein, “Tagg” refers to aggregation temperature indicating the temperature at which protein begins to aggregate.

### Compound of Formula (I′)

In one aspect, provided is a pharmaceutical composition comprising the AIAC, wherein the AIAC comprises the compound of formula (I′) (formula (I′-1) or formula (I′-2) or a mixture thereof):

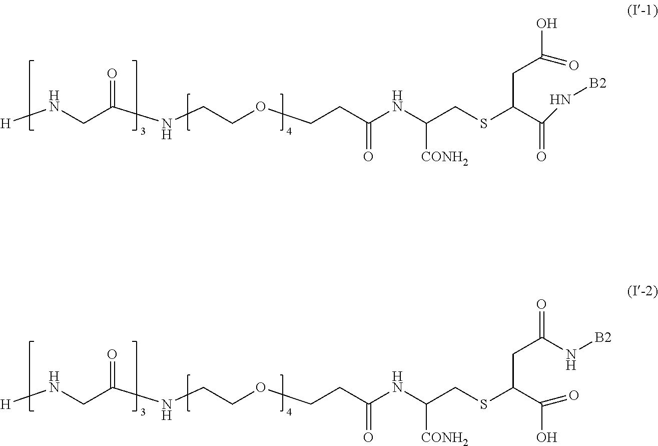

wherein,
B2 is —(CH₂)ₖ(CO)—NH—(C₂H₄—O)ⱼ—R³, or —(CH₂)ₖ(CO)—(NH—CR¹R²—(CO))_{d}—R³;
R¹ is selected from hydrogen, —OH, —NH₂ and —C₁₋₆ alkyl;
R² is selected from hydrogen, —OH, —NH₂ and —C₁₋₆ alkyl;
R³ is a group which can leave when reacting with a group in the payload;
k is each independently an integer of 1 to 5, j is an integer of 1 to 3, d is 1 or 2.

In one embodiment, R¹ and R² are each independently hydrogen or C₁₋₆ alkyl. In a preferred embodiment, R¹ and R² are each independently both hydrogen or both C₁₋₆ alkyl. In a more preferred embodiment, R¹ and R² are both hydrogen.

In one embodiment, B2 in formula (I′-1) and (I′-2) are the same.

In one embodiment, k is 2. In one embodiment, j is 1.

In one embodiment, d is 1.

In one embodiment, the terminal group R³ is hydrogen. In one embodiment, R³ is hydroxy or

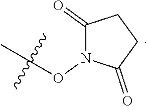 [SMILES: *ON1C(=O)CCC1=O]

In one embodiment, the terminal group R³ represents the part of structure which would not appear in the product molecule resulting from the reaction of B2 with the payload, and thus in the linking unit-payload intermediate (c.f. below) the structure moiety corresponding to B2 is the said one of or the combination of two or more of the bivalent groups.

Thiosuccinimide is unstable under physiological conditions and is liable to reverse Michael addition which leads to cleavage at the conjugation site. Moreover, when another thiol compound is present in the system, thiosuccinimide may also undergo thiol exchange with the other thiol compound. Both of these reactions cause the fall-off of the payload and result in toxic side effects. In the present disclosure, the ring-opened succinimide structure

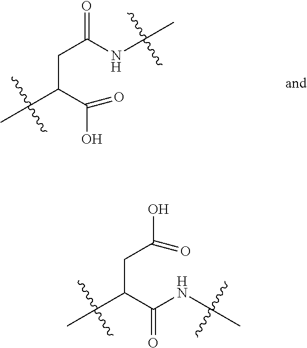 [SMILES: *NC(=O)CC(*)C(=O)O ; *NC(=O)C(*)CC(=O)O]

no longer undergoes reverse Michael addition or thiol exchange, and thus the product is more stable. Method of ring opening reaction can be found in WO2015165413A1.

### Specific Embodiment of the Formula (I′) Compound

In one embodiment, in compound of formula (I′), B2 is —(C_{H}2)ₖ(CO)—NH—(C₂H₄—O)ⱼ—H, k is 2, j is 1, and the structure of the linking unit is a mixture of the following two structures (linking unit LN102-6):

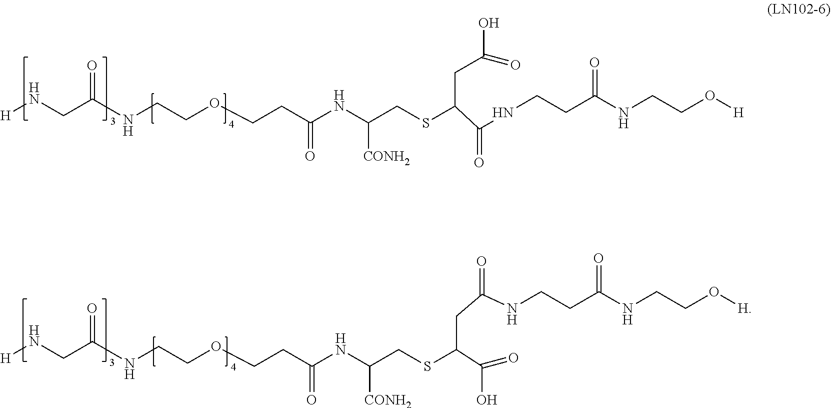

In one embodiment, in compound of formula (I′), B2 is —(CH₂)ₖC(O)—(NH—CR¹R²—C(O))_{d}—R³, k is 2, d is 1, R¹ and R² are hydrogen, and the structure of the linking unit is a mixture of the following two structures (linking unit LN102-8):

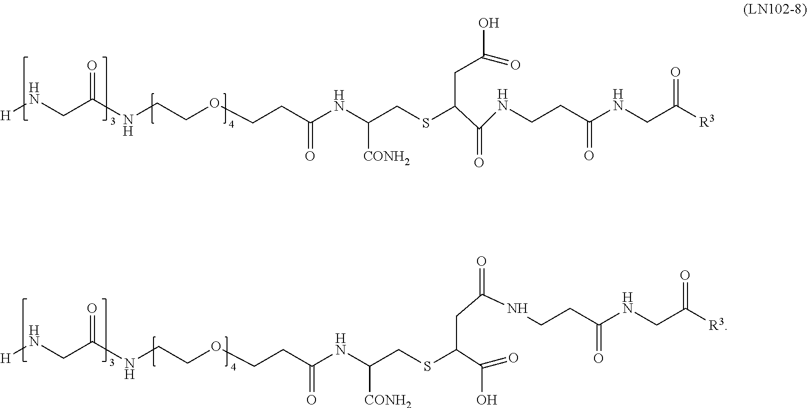

R³ is a group which can leave when reacting with a group in the payload; In one embodiment, R³ is hydroxy or

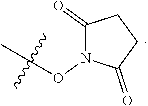 [SMILES: *ON1C(=O)CCC1=O]

It is to be understood that when there are two or more R^{x} (x being 1, 2, 3, 4, 5, 6, 7, etc.), each R^{x} is selected independently. In some embodiments, the “x”s in the molecule are denoted with or without additional apostrophe (′) or apostrophes (such as ″, ′″, ″″, etc.), for example R, R^{1′}, R^{1″}, R^{1′″}, R^{2′}, R^{2″}, R^{2′″}, etc. The other R's such as R³ should be understood in a similar way.

### Compound of Formula (I′) as Linking Unit

In one embodiment, the reactive group comprised by B2 can be used to covalently conjugate with a payload containing another reactive group, such that the compound of formula (I′) bears a payload.

In another embodiment, the ligase recognition sequence GGG (G is glycine) comprised by formula (I′) can be used in the conjugation by a ligase with the corresponding ligase recognition sequence LPETGG (SEQ ID NO: 28).

Thus, a compound of formula (I′) can be used as a linking unit that can be linked to a targeting molecule (such as an antibody or antigen-binding fragment thereof) and/or a payload.

One skilled in the art can synthesize the linking units by conventional solid phase or liquid phase methods.

### Payload-Bearing Formula (I′) Compound

The reactive group comprised by B2 is covalently conjugated with a payload containing another reactive group to give a payload-bearing formula (I′) compound.

In yet another aspect, provided is a pharmaceutical composition comprising an AIAC, wherein the AIAC comprises compound having the structure of formula (II′-1) or (II′-2), or the mixture thereof,

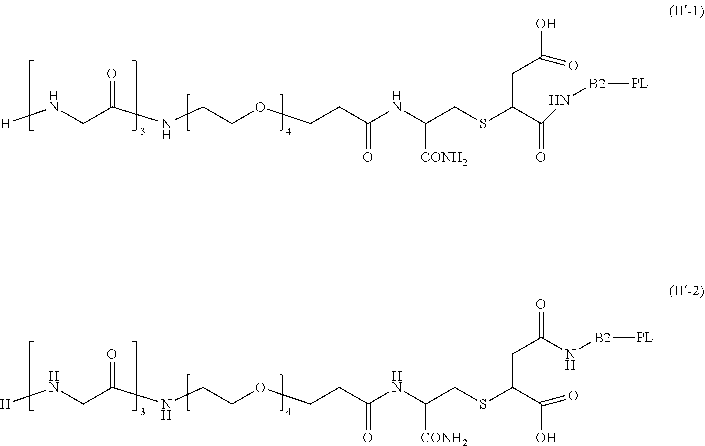

wherein
PL is a Payload which is linked to the B2 moiety of the compound of formula (I′).

### Payload

In the present disclosure, the payload may be selected from small molecule compounds, nucleic acids and analogues, tracer molecules (including fluorescent molecules, etc.), short peptides, polypeptides, peptidomimetics, and proteins. In one embodiment, the payload is selected from small molecule compounds, nucleic acid molecules, and tracer molecules. In a preferred embodiment, the payload is selected from small molecule compounds. In a more preferred embodiment, the payload is selected from cytotoxin and fragments thereof. In a more preferred embodiment, the payload is selected from immune agonist and fragments thereof.

In one embodiment, the immune agonist is selected from TLR agonists such as TLR agonists (e.g., TLR 7 agonists, TLR 8 agonists, TLR 7/8 agonists) and STING agonists. In one embodiment, the immune agonist is selected from TLR agonists.

In one embodiment, the immune agonist is Resiquimod:

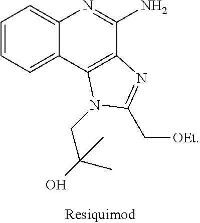 [SMILES: CCOCc1nc2c(N)nc3ccccc3c2n1CC(C)(C)O]

In one embodiment, the linking unit and the Payload are connected via reactive groups as defined above, using any reaction known in the art, including but not limit to condensation reaction, nucleophilic addition, electrophilic addition, etc.

In one embodiment, the payload is an immune agonist, the antibody-immune agonist conjugate (numbered as LPx) is one of the compounds as shown in the following table.

| Compound of formula (II′) | Linking unit | PL (immune agonist) |
|---|---|---|
| LP102-6-1 | LN102-6 | Resiquimod |
| LP102-8-1 | LN102-8 | Resiquimod |

### Preparation of the Payload-Bearing Formula (I′) Compound

In one embodiment, the linking unit and the Payload are connected via reactive groups as defined above, using any reaction known in the art, including but not limit to condensation reaction, nucleophilic addition, electrophilic addition, etc.

### Compound of Formula (III′)

In one aspect, provided is a compound of formula (III′):

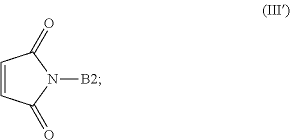

wherein B2 is as defined in formula (I′).

In one embodiment, the compound of formula (III′) could be used to prepare the payload-bearing formula (I′) compound through the following route:

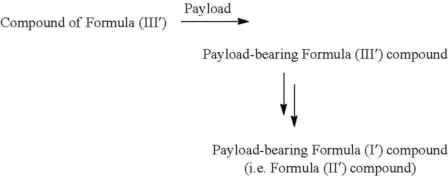

The transformation of Payload-bearing Formula (III′) compound to Payload-bearing Formula (l′) compound could be conducted using any known method in the art or as described herein. For example, single step or multi step synthesis could be conducted to introduce the structure fragment

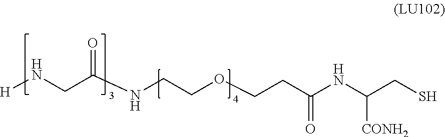

to maleimide ring in the Payload-bearing Formula (III′) compound, and then the resulting molecule which contains a succinimide moiety could undergo ring-opening reaction to open the succinimide ring and obtain the Payload-bearing Formula (I′) compound (i.e. Formula (II′) compound). In one embodiment, LU102 is introduced to the Payload-bearing Formula (III′) compound through the reaction of maleimide group contained in Formula (III′) compound with the thiol group of LU102.

### Conjugates and Preparation Thereof

In yet another aspect, provided is a conjugate having the structure of formula (II-1) and/or (II-2)

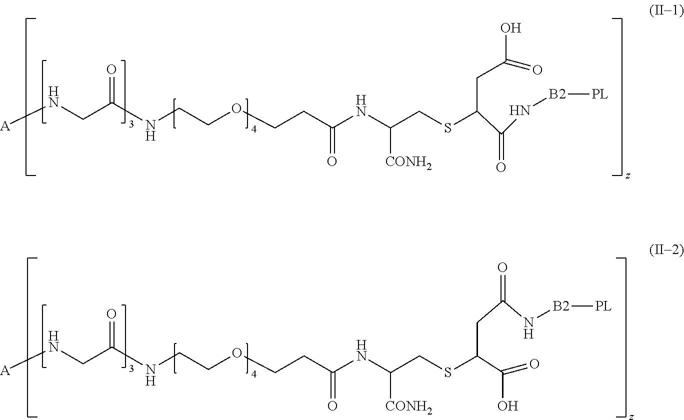

wherein,
PL is a payload which is linked to B2 moiety of the compound of formula (I′);
z is an integer of 1 to 4; preferably 1 to 2;
A is a targeting molecule.

In one embodiment, the payload is an immune agonist, which is as defined above. In one embodiment, the conjugate is an antibody-immune agonist conjugate.

### Targeting Molecule

In one embodiment, the targeting molecule is an antibody or an antigen binding fragment thereof.

In some embodiments of the present disclosure, targets recognized by the targeting molecules (such as an antibody or antigen-binding fragment thereof) is ErbB2/HER2.

In one embodiment, the targeting molecule is an anti-human HER2 antibody or antigen-binding fragment thereof. Examples of anti-human HER2 antibodies include but are not limited to Trastuzumab. Trastuzumab binds to the fourth extracellular domain (ECD4) of HER2 and is approved for the treatment of HER2-positive breast cancer and gastric cancer.

In a preferred embodiment, the anti-human HER2 antibody is one or more selected from engineered anti-HER2 antibodies based on Trastuzumab.

In a preferred embodiment, the anti-human HER2 antibody is a recombinant antibody selected from monoclonal antibody, chimeric antibody, humanized antibody, antibody fragment, and antibody mimic. In one embodiment, the antibody mimic is selected from scFv, minibody, diabody, nanobody. For the conjugation with the compound of formula (I′), formula (II′-1) or formula (II′-2), the targeting molecule of the present disclosure may comprise a modified moiety to connect with the compound of formula (I′), formula (II′-1) or formula (II′-2). The introduction position of such modified moiety is not limited, for example, when the targeting molecule is an antibody, its introduction position can be, but not limited to, located at the C-terminal and/or the N-terminal of the heavy chain and/or light chain of the antibody.

In one embodiment, the targeting molecule of the present disclosure is an antibody or antigen-binding fragment thereof, which may comprise terminal modification. A terminal modification refers to a modification at the C-terminal and/or N-terminal of the heavy chain and/or light chain of the antibody, which for example comprises a ligase recognition sequence. In another embodiment, the terminal modification may further comprise spacer Sp1 comprising 2-10 amino acids, wherein the antibody, Sp1 and the ligase recognition sequence are sequentially linked. In a particular embodiment, Sp1 is a spacer sequence selected from GA, GGGGS (SEQ ID NO: 25), GGGGSGGGGS (SEQ ID NO: 26), GGGGSGGGGSGGGGS (SEQ ID NO: 27), especially GA.

In a preferred embodiment, the light chain of the antibody or antigen-binding fragment thereof includes 3 types: wild-type (LC); the C-terminus modified light chain (LCCT), which is modified by direct introduction of a ligase recognition sequence LPETGG and C-terminus modified light chain (LCCT_{L}), which is modified by introduction of short peptide spacers plus the ligase donor substrate recognition sequence LPETGG. The heavy chain of the antibody or antigen-binding fragment thereof includes 3 types: wild-type (HC); the C-terminus modified heavy chain (HCCT), which is modified by direct introduction of a ligase recognition sequence LPETGG; and C-terminus modified heavy chain (HCCT_{L}), which is modified by introduction of short peptide spacers plus the ligase donor substrate recognition sequence LPETGG. When z in the compound of formula (II-1) and/or formula (II-2) is 1 or 2, the combination of the above heavy and light chains can form 8 preferred antibody molecules, see the amino acid sequence table.

In one embodiment, the targeting molecule of the present disclosure is an antibody comprising a V_{L} and a V_{H}, wherein the V_{L} comprises LCDR1 having the amino acid sequence of SEQ ID NO: 17 (RASQDVNTAVA), LCDR2 having the amino acid sequence of SEQ ID NO: 18 (SASFLYS), and LCDR3 having the amino acid sequence of SEQ ID NO: 19 (QQHYTTPPT), wherein the V_{H} comprises HCDR1 having the amino acid sequence of SEQ ID NO: 20 (DTYIH), HCDR2 having the amino acid sequence of SEQ ID NO: 21 (RIYPTNGYTRYADSVKG), and HCDR3 having the amino acid sequence of SEQ ID NO: 22 (WGGDGFYAMDY). In one embodiment, the antibody is modified by introduction of the ligase donor substrate recognition sequence. In one embodiment, the antibody comprises a V_{L} having the amino acid sequence of SEQ ID NO: 23 and a V_{H} having the amino acid sequence of SEQ ID NO: 24.

In one embodiment, the sequences of CDR and Variable domain are defined according to Kabat numbering system. In one embodiment, the targeting molecule of the present disclosure is an antibody comprising a light chain having the amino acid sequence of SEQ ID NO: 1 and a heavy chain having the amino acid sequence of SEQ ID NO: 2. In one embodiment, the targeting molecule of the present disclosure is an antibody comprising a light chain having the amino acid sequence of SEQ ID NO: 3 and a heavy chain having the amino acid sequence of SEQ ID NO: 4. In one embodiment, the targeting molecule of the present disclosure is an antibody comprising a light chain having the amino acid sequence of SEQ ID NO: 5 and a heavy chain having the amino acid sequence of SEQ ID NO: 6. In one embodiment, the targeting molecule of the present disclosure is an antibody comprising a light chain having the amino acid sequence of SEQ ID NO: 7 and a heavy chain having the amino acid sequence of SEQ ID NO: 8. In one embodiment, the targeting molecule of the present disclosure is an antibody comprising a light chain having the amino acid sequence of SEQ ID NO: 9 and a heavy chain having the amino acid sequence of SEQ ID NO: 10. In one embodiment, the targeting molecule of the present disclosure is an antibody comprising a light chain having the amino acid sequence of SEQ ID NO: 11 and a heavy chain having the amino acid sequence of SEQ ID NO: 12. In one embodiment, the targeting molecule of the present disclosure is an antibody comprising a light chain having the amino acid sequence of SEQ ID NO: 13 and a heavy chain having the amino acid sequence of SEQ ID NO: 14. In one embodiment, the targeting molecule of the present disclosure is an antibody comprising a light chain having the amino acid sequence of SEQ ID NO: 15 and a heavy chain having the amino acid sequence of SEQ ID NO: 16.

The conjugates of the present disclosure can further comprise a payload. The payload is as described above.

### Specific Embodiments for the Conjugate

In one embodiment, in formula (II-1) and/or formula (II-2), B2 is —(CH₂)ₖ(CO)—NH—(C₂H₄—O)ⱼ—, k is 2, j is 1, and the structure of the conjugate is as follows (formula AC102-6):

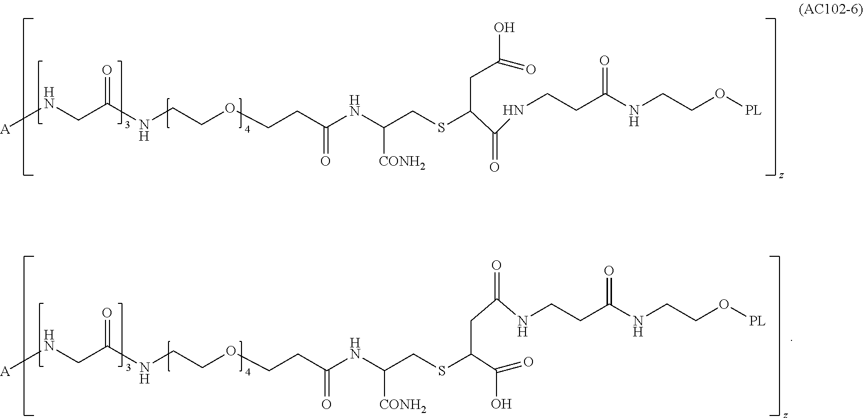

In one embodiment, in formula (II-1) and/or formula (II-2), B2 is —(CH₂)ₖC(O)—(NH—CR¹R²—C(O))_{d}—, k is 2, d is 1, R¹ and R² are hydrogen, and the structure of the conjugate is as follows (formula AC102-8):

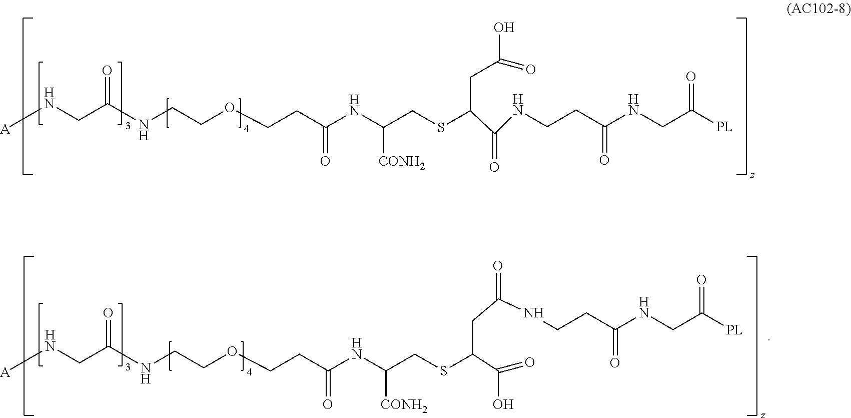

### Preparation of the Conjugate

The conjugates of the present disclosure can be prepared by any method known in the art. In some embodiments, the conjugate is prepared by the ligase-catalyzed site-specific conjugation of a targeting molecule and a payload-bearing formula (I′) compound, wherein the targeting molecule is modified by a ligase recognition sequence, such as ligase donor substrate recognition sequence. The method comprises step A and step B.

Step a. Preparation of the Linking Unit-Payload Intermediate

In a preferred embodiment, B2 in the compound of formula (I′) is covalently linked via a reactive group to a payload containing a corresponding reactive group, wherein the reactive groups are respectively as defined above.

The linking unit-payload intermediate prepared using the compound of formula (I′) of the present disclosure has defined structure, defined composition and high purity, so that when the conjugation reaction with an antibody is conducted, fewer impurities are introduced or no other impurities are introduced. When such an intermediate is used for the ligase-catalyzed site-specific conjugation with a modified antibody containing a ligase recognition sequence, a homogeneous ADC with highly controllable quality is obtained.

### Step B. Linking the Targeting Molecule to the Payload-Bearing Formula (I′) Compound

The targeting molecule of the present disclosure can be conjugated with the payload-bearing formula (I′) compound (i.e., the compound of formula (II′) by any method known in the art. For example, ligase-catalyzed site-specific conjugation technique is applied, and the targeting molecule and the payload-bearing formula (I′) compound are linked to each other via the ligase-specific recognition sequences of the substrates. In one embodiment, the targeting molecule is an antibody with recognition sequence-based terminal modifications introduced at the C-terminal of the light chain and/or the heavy chain, and the targeting molecule is conjugated with the compound of formula (II′), under the catalysis of the wild type or optimized engineered ligase or any combination thereof, and under suitable catalytic reaction conditions.

In a specific embodiment, the ligase is Sortase A and the conjugation reaction can be represented by the following scheme:

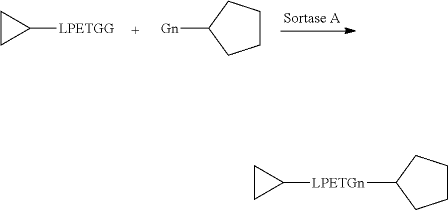

The triangle and pentagon respectively represent any of the following: a portion of an antibody or a portion of a compound of formula (II′). N is respectively as defined above. When conjugated with Gₙ, which is the corresponding recognition sequence of the acceptor substrate, the upstream peptide bond of the glycine in the LPETGG sequence is cleaved by Sortase A, and the resulting intermediate is linked to the free N-terminal of G, to generate a new peptide bond. The resulting amino acid sequence is LPETGₙ (SEQ ID NO: 30). The sequences Gₙ and LPETGG are as defined above.

### Table of Specific Conjugates

In one embodiment, the payload is an immune agonist. In one embodiment, the antibody-immune agonist conjugate is as shown in the following table:

| | | PL (immune | A (targeting |
|---|---|---|---|
| AIAC | Formula | agonist) | molecule) |
| AC102-6-1-1 | AC102-6 | Resiquimod | Ab0001-LCCT_{L}-HC |
| AC102-8-1-1 | AC102-8 | Resiquimod | Ab0001-LCCT_{L}-HC |

### Pharmaceutical Composition and Pharmaceutical Preparation

Another aspect of the disclosure is to provide a pharmaceutical composition comprising a prophylactically or therapeutically effective amount of a conjugate of the present disclosure, and at least one pharmaceutically acceptable carrier.

The pharmaceutical composition of the present disclosure may be administered in any manner as long as it achieves the effect of preventing, alleviating, preventing or curing the symptoms of a human or animal. For example, various suitable dosage forms can be prepared according to the administration route, especially injections such as lyophilized powder for injection, injection, or sterile powder for injection.

The term “pharmaceutically acceptable” means that when contacted with tissues of the patient within the scope of normal medical judgment, no undue toxicity, irritation or allergic reaction, etc. shall arise, having reasonable advantage-disadvantage ratios and effective for the intended use.

In one embodiment, the pharmaceutical composition of the present disclosure has a drug to antibody ratio (DAR) of an integer or non-integer of 1 to 4, such as about 1-4, about 1-3.5, about 1-3, about 1-2.5, preferably about 1-2. In one embodiment, the pharmaceutical composition of the present disclosure has a DAR of about 1.5-about 2, preferably about 1.6-about 2, more preferably about 1.7-about 2.

In one embodiment, the pharmaceutical composition comprises an AIAC, buffer, and one or more stabilizers.

In one embodiment, the buffer in the pharmaceutical composition comprises one or more of Sodium Acetate, Sodium Citrate, Sodium Phosphate, Histidine, Tris and Glycine. In some embodiments, the buffer is selected from citrate buffer, phosphate buffer, histidine buffer and acetate buffer. In some embodiments, the citrate buffer comprises citric acid and sodium citrate, the histidine buffer comprises L-Histidine and L-histidine hydrochloride, and the acetate buffer comprises acetic acid and sodium acetate. In another embodiment, the buffer comprises succinate buffer. In one embodiment, the succinate buffer comprises succinic acid and sodium succinate.

In some embodiment, the concentration of buffer is about 10-40 mM. In a preferred embodiment, the concentration of buffer is about 10 mM, about 14 mM, about 18 mM, about 19 mM, about 20 mM, about 21 mM, about 22 mM, about 23 mM, about 26 mM, about 30 mM, about 33 mM, about 37 mM or about 40 mM. In a preferred embodiment, the concentration of buffer is about 20 mM. In some embodiment, the concentration of buffer is about 15-25 mM. In a preferred embodiment, the concentration of buffer is about 20 mM. In one embodiment, the buffer in the pharmaceutical composition comprises one or more of about 15-25 mM Sodium Acetate, Sodium Citrate, Sodium Phosphate, Histidine, Tris and Glycine. In some embodiments, the concentration of buffer is about 15 to 25 mM. In some embodiments, the buffer comprises about 15-25 mM citrate buffer, about 15-25 mM phosphate buffer, about 15-25 mM histidine buffer and about 15-25 mM acetate buffer.

In one embodiment, the buffer in the pharmaceutical composition comprises one or more of Sodium Acetate, Sodium Citrate and Histidine. In some embodiment, the buffer comprises one or more of citrate buffer, histidine buffer and acetate buffer. In some embodiments, the buffer is citrate buffer, histidine buffer or acetate buffer.

In one embodiment, the buffer in the pharmaceutical composition comprises one or more of about 15-25 mM Sodium Acetate, Sodium Citrate and Histidine.

In one embodiment, the buffer in the pharmaceutical composition comprises about 15-25 mM Histidine. In some embodiments, the buffer is about 15-25 mM histidine buffer, such as about 15 mM, about 16.5 mM, about 17 mM, about 18.3 mM, about 19 mM, about 21 mM, about 22 mM, about 23 mM or about 25 mM.

In one embodiment, the buffer in the pharmaceutical composition comprises about 20 mM Histidine. In some embodiments, the buffer in the pharmaceutical composition is about 20 mM histidine buffer.

In one embodiment, the pH of the buffer in the pharmaceutical composition is about 4.0-6.0, such as about 4.0, about 4.4, about 4.8, about 5.0, about 5.2, about 5.4, about 5.7 or about 6.0.

In one embodiment, the pH of the buffer in the pharmaceutical composition is about 5.5.

In one embodiment, the stabilizers in the pharmaceutical composition comprise one or more of sucrose, trehalose dihydrate, trehalose and sorbitol.

In one embodiment, the stabilizers in the pharmaceutical composition comprise one or more of about 5-15% (W/V) sucrose, trehalose dihydrate, trehalose and sorbitol. In some embodiments, the concentration of stabilizer is about 5%, 5.8%, 6.5%, 7.3%, 7.9%, 8.2%, 8.5%, 9%, 9.9%, 10%, 11%, 12%, 13%, 14% or 15% (W/V).

In one embodiment, the stabilizers in the pharmaceutical composition comprise one or more of about 9% (W/V) sucrose, trehalose dihydrate, trehalose and sorbitol.

In one embodiment, the stabilizers in the pharmaceutical composition comprise sucrose.

In one embodiment, the stabilizers in the pharmaceutical composition comprise about 5-15% (W/V) sucrose. In some embodiments, the concentration of sucrose is about 5%, 5.8%, 6.5%, 7.3%, 7.9%, 8.2%, 8.5%, 9%, 9.9%, 10%, 11%, 12%, 13%, 14% or 15%.

In one embodiment, the stabilizers in the pharmaceutical composition comprise about 9% (W/V) sucrose.

In some embodiments, the pharmaceutical further comprises surfactant. In some embodiments, the surfactant is tween 20 (PS20) and/or tween 80 (PS80). In some embodiments, the concentration of surfactant is about 0.05-0.5 mg/ml. In one embodiment, the surfactant in the pharmaceutical composition comprises about 0.05-0.5 mg/ml PS 20. In some embodiments, the concentration of PS 20 is about 0.05 mg/ml, about 0.1 mg/ml, about 0.15 mg/ml, about 0.2 mg/ml, about 0.24 mg/ml, about 0.25 mg/ml, about 0.3 mg/ml, about 0.37 mg/ml, about 0.4 mg/ml, about 0.48 mg/ml or about 0.5 mg/ml.

In one embodiment, the surfactant in the pharmaceutical composition comprises 0.2 mg/ml PS 20. In another embodiment, the surfactant in the pharmaceutical composition comprises 0.4 mg/ml PS 20.

In some embodiment, the pharmaceutical composition comprises: about 10-40 mM buffer, pH 4.0-6.0; about 5-15% (W/V) stabilizer; about 0.05-0.5 mg/ml surfactant. In some embodiments, the pharmaceutical composition comprises: about 15-25 mM buffer, pH 4.0-6.0; about 5-15% (W/V) stabilizer; about 0.05-0.5 mg/ml surfactant. In one embodiment, the pharmaceutical composition comprises 10-40 mM histidine buffer, pH 4.0-6.0; 5-15% (W/V) sucrose; 0.05-0.5 mg/ml PS 20. In one embodiment, the pharmaceutical composition comprises 15-25 mM histidine buffer, pH 4.0-6.0; 5-15% (W/V) sucrose; 0.05-0.5 mg/ml PS 20.

In one embodiment, the pharmaceutical composition comprises about 20 mM histidine buffer, pH about 5.5; about 9% (W/V) sucrose; about 0.2 mg/ml PS 20.

In one embodiment, the concentration of the protein of AIAC in the pharmaceutical composition is about 18-110 mg/ml, such as about 18 mg/ml, about 19 mg/ml, about 20 mg/ml, about 24 mg/ml, about 27 mg/ml, about 30 mg/ml, about 37 mg/ml, about 40 mg/ml, about 47 mg/ml, about 50 mg/ml, about 55 mg/ml, about 60 mg/ml, about 62 mg/ml, about 66 mg/ml, about 70 mg/ml, about 75 mg/ml, about 80 mg/ml, about 82 mg/ml, about 85 mg/ml, about 90 mg/ml, about 95 mg/ml, about 100 mg/ml, about 105 mg/ml or about 110 mg/ml. In a preferred embodiment, the concentration of the protein of AIAC in the pharmaceutical composition is about 62.2 mg/ml. In another preferred embodiment, the concentration of the protein of AIAC in the pharmaceutical composition is about 82.2 mg/ml. In another preferred embodiment, the concentration of the protein of AIAC in the pharmaceutical composition is about 100.4 mg/ml.

In one embodiment, the concentration of the protein of AIAC in the pharmaceutical composition is about 18 mg/ml-66 mg/ml, such as about 18 mg/ml, about 19 mg/ml, about 20 mg/ml, about 24 mg/ml, about 27 mg/ml, about 30 mg/ml, about 37 mg/ml, about 40 mg/ml, about 47 mg/ml, about 50 mg/ml, about 55 mg/ml, about 60 mg/ml or about 66 mg/ml. In one embodiment, the concentration of the AIAC in the pharmaceutical composition is about 18 mg/ml-66 mg/ml, such as about 18 mg/ml, about 19 mg/ml, about 20 mg/ml, about 24 mg/ml, about 27 mg/ml, about 30 mg/ml, about 37 mg/ml, about 40 mg/ml, about 47 mg/ml, about 50 mg/ml, about 55 mg/ml, about 60 mg/ml or about 66 mg/ml.

In one embodiment, the concentration of the protein of AIAC in the pharmaceutical composition is about 20 mg/ml-60 mg/ml. In one embodiment, the concentration of the AIAC in the pharmaceutical composition is about 20 mg/ml-60 mg/ml.

In one embodiment, the concentration of the protein of AIAC in the pharmaceutical composition is about 20 mg/ml. In one embodiment, the concentration of the AIAC in the pharmaceutical composition is about 20 mg/ml.

In one embodiment, the concentration of the protein of AIAC in the pharmaceutical composition is about 40 mg/ml. In one embodiment, the concentration of the AIAC in the pharmaceutical composition is about 40 mg/ml.

In one embodiment, the concentration of the protein of AIAC in the pharmaceutical composition is about 60 mg/ml. In one embodiment, the concentration of the AIAC in the pharmaceutical composition is about 60 mg/ml.

In some embodiments, the formulation comprises: 20 mg/ml protein of AIAC, 20 mM histidine buffer (pH 5.5), 9% sucrose, and 0.2 mg/ml tween 20. In some embodiments, the formulation comprises: 40 mg/ml protein of AIAC, 20 mM histidine buffer (pH 5.5), 9% sucrose, and 0.2 mg/ml tween 20. In some embodiments, the formulation comprises: 60 mg/ml protein of AIAC, 20 mM histidine buffer (pH 5.5), 5% sucrose, and 0.2 mg/ml tween 20.

In some embodiments, the formulation comprises: 62.2 mg/ml protein of AIAC, 20 mM histidine buffer (pH 5.65), 7.5% sucrose, and 0.2 mg/ml tween 20. In some embodiments, the formulation comprises: 82.2 mg/ml protein of AIAC, 20 mM histidine buffer (pH 5.68), 7.5% sucrose, and 0.2 mg/ml tween 20. In some embodiments, the formulation comprises: 100.4 mg/ml protein of AIAC, 20 mM histidine buffer (pH 5.69), 7.5% sucrose, and 0.2 mg/ml tween 20. In some embodiments, the formulation comprises: 62.2 mg/ml protein of AIAC, 20 mM histidine buffer (pH 5.65), 9% sucrose, and 0.2 mg/ml tween 20. In some embodiments, the formulation comprises: 82.2 mg/ml protein of AIAC, 20 mM histidine buffer (pH 5.68), 9% sucrose, and 0.2 mg/ml tween 20. In some embodiments, the formulation comprises: 100.4 mg/ml protein of AIAC, 20 mM histidine buffer (pH 5.69), 9% sucrose, and 0.2 mg/ml tween 20.

In some embodiments, the AIAC has the structure of formular (II-1), (II-2) or the mixture thereof. In some embodiments, the AIAC is AC102-6-1-1. In some embodiments, the AIAC is AC102-8-1-1.

### Treatment Method and Use

The conjugates of the present disclosure are useful for the treatment of tumors and/or autoimmune diseases. Tumors susceptible to conjugate treatment include those characterized by specific tumor-associated antigens or cell surface receptors, and those will be recognized by the targeting molecule in the conjugate and can be affected by the immune cell activation activity of agonist in the conjugate.

Accordingly, in yet another aspect, also provided is use of a conjugate of the present disclosure or a pharmaceutical composition of the present disclosure in the manufacture of a medicament for preventing, alleviating or treating a disease, disorder or condition selected from a tumor or an autoimmune disease.

In another aspect, provided is a conjugate of the present disclosure or a pharmaceutical composition of the present disclosure for use in the prevention, alleviation or treatment of a tumor or an autoimmune disease.

In a further aspect, provided is a method of preventing, alleviating or treating a tumor or an autoimmune disease, the method comprising administering to an individual in need thereof an effective amount of a conjugate of the present disclosure or a pharmaceutical composition of the present disclosure.

In a preferred embodiment, the conjugate of the present disclosure formed by conjugation of the anti-human HER2 antibody and the payload can specifically bind to HER2 on the surface of the tumor cell and selectively kill the HER2-expressing tumor cells. In another preferred embodiment, provided is use of a conjugate of the present disclosure or a pharmaceutical composition of the present disclosure in the manufacture of a medicament for preventing, alleviating or treating a disease, disorder or condition selected from HER2-positive tumors. In a more preferred embodiment, the disease, disorder or condition is selected from breast cancer, gastric cancer, lung cancer, ovarian cancer, urothelial cancer, and the like.

The dosage of the conjugate administered to the subject can be adjusted to a considerable extent. The dosage can vary according to the particular route of administration and the needs of the subject, and can be subjected to the judgment of the health care professional.

### Beneficial Effect

The present disclosure utilizes a linking unit with unique structure and uses a ligase to catalyze the conjugation of the antibody (i.e., anti-HER2 antibody) and the agonist. The conjugate of the present disclosure has good homogeneity, high activity and high selectivity. In particular, the intracellular metabolites show significantly reduced cell proliferation toxicities to the cells with low expression or no expression of target antigens. Furthermore, the toxicity of the linking unit-agonist intermediate is much lower than that of the free agonist, and thus the manufacture process of the drug is less detrimental, which is advantageous for industrial production. And the formulation of the present disclosure ensures that the ADCs and AIACs of the present disclosure have good physicochemical properties and biological properties.

The conjugate of the present disclosure achieves at least one of the following technical effects:
(1) High inhibitory activity against target cells, or strong killing effect on target cells.
(2) Good physicochemical properties (e.g., solubility, physical and/or chemical stability).
(3) Good pharmacokinetic properties (e.g., good stability in plasma, appropriate half-life and duration of action).
(4) Good safety (low toxicity on non-target normal cells or tissues, and/or fewer side effects, wider treatment window), etc.

The drug can prevent the patient from resisting to HER2-targeting therapy, and activate myeloid cells to enhance innate and adaptive immune response. It can overcome low response rate of current HER2-directed therapies. And the formulation of the present disclosure can ensure at least one or more of the above technical effects of the AIACs of the present disclosure.

### EXAMPLES

### Preparation Example

In order to more clearly illustrate the objects and technical solutions, the present disclosure is further described below with reference to specific examples. It is to be understood that the examples are not intended to limit the scope of the disclosure. The specific experimental methods which were not mentioned in the following examples were carried out according to conventional experimental method.

### Instruments, Materials and Reagents

Unless otherwise stated, the instruments and reagents used in the examples are commercially available. The reagents can be used directly without further purification.
MS: Thermo Fisher Q Exactive Plus, Water2795-Quattro micro triple quadrupole mass spectrometer
HPLC: Waters 2695, Agilent 1100, Agilent 1200
Semi-preparative HPLC: Lisure HP plus 50D
Flow Cytometry: CytoFLEX S
HIC-HPLC: Butyl-HIC; mobile phase A: 25 mM PB, 2M (NH₄)₂SO₄, pH 7.0; mobile phase B: 25 mM PB, pH 7.0; flow rate: 0.8 ml/min; acquisition time: 25 min; injection amount: 20 μg; column temperature: 25° C.; detection wavelength: 280 nm; sample chamber temperature: 8° C.
SEC-HPLC: column: TSK-gel G3000 SWXL, TOSOH 7.8 mm ID×300 mm, 5 μm; mobile phase: 0.2 M KH₂PO₄, 0.25 M KCl, pH 6.2; flow rate: 0.5 ml/min; acquisition time: 30 min; injection volume: 50 μl; column temperature: 25° C.; detection wavelength; 280 nm; sample tray temperature: 8° C.
CHO was obtained from Thermo Fisher Scientific; pcDNA 3.3 was obtained from Life Technology; HEK293F was obtained from Prejin; PEIMAX transfection reagent was obtained from Polyscience; MabSelect Sure ProA was obtained from GE; Capto S ImpAct was obtained from GE; Rink-amide-MBHA-resin and dichloro resin were obtained from Nankai synthesis; HCC1954 was obtained from ATCC CAT #CRL-2338; SK-BR-3 was obtained from ATCC CAT #HTB-30; BT474 cells was obtained from ATCC CAT #HTB-20; JIMT1 cells was obtained from DSMZ CAT #ACC589; Colo205 cells was obtained from ATCC CAT #CRL-222; MC38Hher2 murine colorectal cancer cells was obtained from Biocytogen; NUGC4 human gastric cancer cells was obtained from JCRB CAT #JCRB0834; NCI-N87 cells (ATCC CAT #CRL-5822); MDA-MB-468 was obtained from ATCC CAT #HTB-132.

### Example 1 Construction of Antibody Expression Vector, Antibody Expression, Purification and Identification

### 1.1 Production of the Modified Anti-Human HER2 Antibody Ab0001-LCCT_{L}-HC

The expression plasmids for antibody Ab0001-LCCT_{L}-HC (light chain SEQ ID NO: 1, heavy chain: SEQ ID NO: 2) were constructed as follows. The sequence of the antibody Ab0001-LCCT_{L}-HC: based on the amino acid sequence of Trastuzumab, and GALPETGG (SEQ ID NO: 29) was introduced at the C-terminal of the light chain, wherein LPETGG is the recognition sequence of the ligase donor substrate, and GA is a spacer sequence. The plasmids were transfected into CHO cells and the cell population was established and screened for a highly expressed cell population, which was cultured with reference to the culture process of Trastuzumab in a 5-10 L reactor, and the supernatant was collected.

### 1.2 The Purification of Antibody Ab0001-LCCT_{L}-HC

The purification of Ab0001-LCCT_{L}-HC was carried out in a standard process using the combination of MabSelect affinity chromatography and Sepharose S cation exchange chromatography, the purified products were dissolved in the original Trastuzumab drug buffer (5 mM histidine-HCl, 2% trehalose, 0.009% Polysorbate 20, PH 6.0), and frozen in small aliquots.

### 1.3 The Quality Control of Antibody Ab0001-LCCT_{L}-HC

The purity of the above purified antibody Ab0001-LCCT_{L}-HC is 98.5% by SDS-PAGE; the content of high molecular weight polymer of the sample is less than 0.4% by SEC-HPLC; endotoxin content is less than 0.098 EU/mg.

### 1.4 Preparation of Other Modified Anti-Human Antibodies

According to a similar method, a terminal modification based on the ligase recognition sequence was introduced at the C-terminal of the light and/or heavy chain of the Trastuzumab, respectively, giving a modified antibody.

The modified anti-human HER2 antibodies based on Ab0001 (Trastuzumab) are listed in Table 1. LPETGG in the terminal modification sequence is a recognition sequence of the ligase donor substrate, and GA is a spacer sequence.

TABLE 1
Modified anti-human HER2 antibodies

| | | Sequence introduced |
|---|---|---|
| | Sequence | at the terminal |
| Ab0001-LCCT_{L}-HC light chain | SEQ ID NO: 1 | GALPETGG |
| Ab0001-LCCT_{L}-HC heavy chain | SEQ ID NO: 2 | —* |
| Ab0001-LC-HCCT light chain | SEQ ID NO: 3 | — |
| Ab0001-LC-HCCT heavy chain | SEQ ID NO: 4 | LPETGG |
| Ab0001-LC-HCCT_{L} light chain | SEQ ID NO: 5 | — |
| Ab0001-LC-HCCT_{L} heavy chain | SEQ ID NO: 6 | GALPETGG |
| Ab0001-LCCT-HC light chain | SEQ ID NO: 7 | LPETGG |
| Ab0001-LCCT-HC heavy chain | SEQ ID NO: 8 | — |
| Ab0001-LCCT-HCCT light chain | SEQ ID NO: 9 | LPETGG |
| Ab0001-LCCT-HCCT heavy | SEQ ID NO: 10 | LPETGG |
| chain | | |
| Ab0001-LCCT-HCCT_{L} light | SEQ ID NO: 11 | LPETGG |
| chain | | |
| Ab0001-LCCT-HCCT_{L} heavy | SEQ ID NO: 12 | GALPETGG |
| chain | | |
| Ab0001-LCCT_{L}-HCCT light | SEQ ID NO: 13 | GALPETGG |
| chain | | |
| Ab0001-LCCT_{L}-HCCT heavy | SEQ ID NO: 14 | LPETGG |
| chain | | |
| Ab0001-LCCT_{L}-HCCT_{L} light | SEQ ID NO: 15 | GALPETGG |
| chain | | |
| Ab0001-LCCT_{L}-HCCT_{L} heavy | SEQ ID NO: 16 | GALPETGG |
| chain | | |

*: “-” indicates no terminal modification.

The CDR sequences, V_{L} and V_{H} of the modified anti-human HER2 antibodies based on Ab0001 (Trastuzumab) are listed in Table 2.

TABLE 2
CDR, V_{L} and V_{H} sequences of the modified
anti-human HER2 antibodies

| | Sequence |
|---|---|
| LCDR1 | SEQ ID NO: 17 |
| LCDR2 | SEQ ID NO: 18 |
| LCDR3 | SEQ ID NO: 19 |
| HCDR1 | SEQ ID NO: 20 |
| HCDR2 | SEQ ID NO: 21 |
| HCDR3 | SEQ ID NO: 22 |
| V_{L} | SEQ ID NO: 23 |
| V_{H} | SEQ ID NO: 24 |

### Example 2 Preparation of Intermediates 2.1 Preparation of the Linking Unit

### Linking Unit Presents

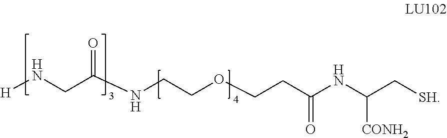

The linking unit fragment LU102 was synthesized by a conventional solid phase polypeptide synthesis using Rink-amide-MBHA-resin or dichloro-resin. Fmoc was used to protect the amino acid in the linking unit. The conjugation reagent was selected from HOBT, HOAt/DIC, DCC, EDCI or HATU. After synthesis, the resin was cleaved using trifluoroacetic acid. The product was purified by HPLC, lyophilized and stored for use. The linking unit fragments are listed in the following table.

| Fragment | Sequence | Mass spectrometry |
|---|---|---|
| LU102 | GGG-NH-(C₂H₄-O)₄-C₂H₄-CO- | 539.2 [M + H]⁺ |
| | Cys-NH₂ | (Theoretical molecular weight: 538.24) |

The linking unit fragments in the above table were reacted with a linking unit fragment which contains a maleimide structure or derivative thereof, and then underwent ring-opening reaction using the method as described in WO2015165413A1 to obtain the linking units LN102-6, LN102-8. Their structures are as shown hereinabove. The Linking unit fragments are listed in the following table:

| Linking unit | Fragment 1 | Fragment 2 |
|---|---|---|
| LN102-6 | LU102 | Mal-NH—C₂H₄—O—H |
| LN102-8 | LU102 | Mal-(NH—CR¹R²—C(O))_{d}—R³ |
| | | R¹═R²═hydrogen |

wherein, Mal is the structural of

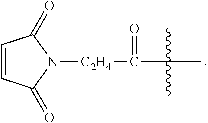

When Fragment 1 and Fragment 2 react to form Linking unit, the maleimide ring of Fragment 2 opens to form the ring-opened succinimide structure

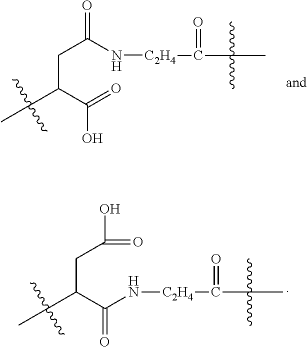

### 2.2 Preparation of Linking Unit-Agonist Intermediates

### 2.2.1 Preparation of Linking Unit-Agonist Intermediates LP102-6-1

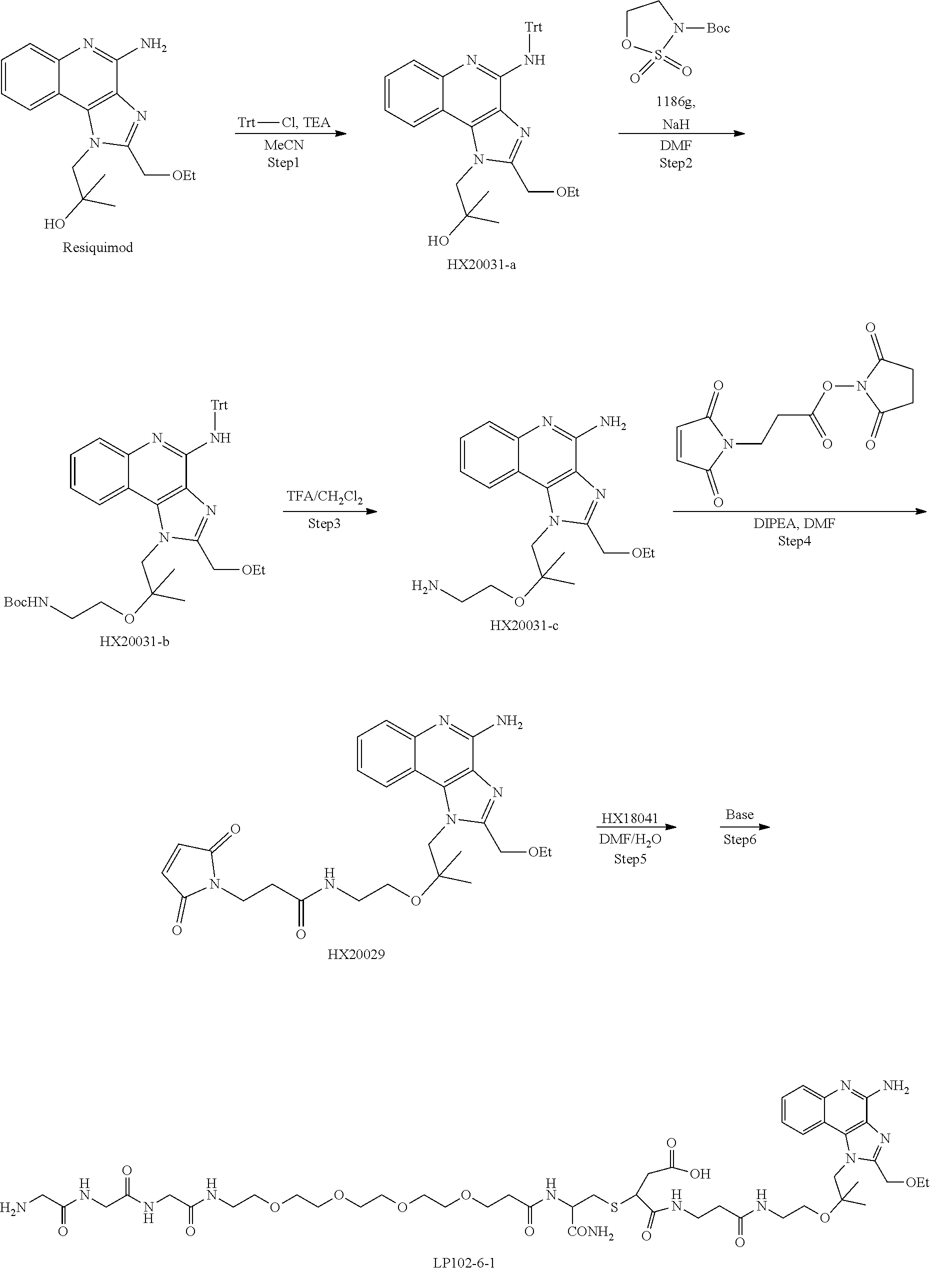

Step 1: Resiquimod (25.0 g, 79.5 mmol) was dissolved in MeCN (500 mL) and treated with Trt-CI (33.25 g, 119.3 mmol) followed by TEA (20.12 mL, 20.12 mmol). The reaction was refluxed for 2-3 h (TLC). The reaction mixture was concentrated in vacuo. Then the mixture was treated with AcOEt (700 mL) and with H₂O (400 mL), stirred for 30 min and separated. The organic phase was concentrated in vacuo to 300 mL and treated with n-heptane (400 mL). Then the mixture was stirred for 20 min. After filtration, the cake was beat with EtOH/H₂O (1:1, 200 mL) and filtrated. The cake was dried in vacuo to obtain target compound HX20031-a was obtained as a white solid (43.9 g, 99.1%).
Step 2: The compound (HX20031-a) (40.02 g, 72.9 mmol) was dissolved in DMF (200 mL) and cooled to 0-10° C. NaH (60%, 3.74 g, 93.4 mmol) was added in batches. The suspension was stirred vigorously at 0-10° C. for 1 h and then warmed to 20-30° C. to stir for 1 h. Then the mixture was cooled to 0-10° C. and treated with compound 1186g (20.88 g, 93.4 mmol) in one portion. The mixture was stirred overnight at room temperature and then treated with the mixture of 10% NaH₂PO₄ (1 L) and AcOEt (1 L) slowly. The reaction mixture was stirred for 3 h and filtrated. The organic layer was concentrated and purified by silica gel column chromatography (n-heptane to n-heptane/AcOEt=10:1 to n-heptane/AcOEt=4:1) to obtain target compound HX20031-b (24.89 g, 46.9%).
Step 3: Compound HX20031-b (10 g, 14.3 mmol) was treated with the mixture of TFA (40 mL) and H₂O (80 mL). The reaction mixture was stirred for 24 h at room temperature. Then the mixture was poured into MTBE (400 mL) and stirred for 2 h. After filtration, the cake was beat with MTBE (200 mL) and filtrated. The cake was dried in vacuo to obtain target compound HX20031-c was obtained as a white solid (8.51 g, 100%).
Step 4: Compound HX20031-c (6.0 g, 10.2 mmol) was dissolved in DMF (50 mL) and treated with DIPEA (3.5 mL, 20.4 mmol) and N-Succinimidyl 3-maleimidopropionate (3.27 g, 12.3 mmol). The reaction was kept at room temperature for 3 h (HPLC), then the mixture was used for next step directly.
Step 5-6: The mixture from step 4 was treated with the solution of Fragment HX18041 (LU102) (5.5 g, 15.3 mmol) and H₂O (50 mL). The mixture was reacted at 0-40° C. for 0.5-20 h. Then the reaction mixture was mixed with an appropriate amount of Tris Base solution or other solution that promotes the ring-opening reaction, and the reaction was performed at 0-40° C. for 0.2-20 h. After the reaction was completed, the product was purified by semi-preparative/preparative HPLC and lyophilized to obtain linking unit-agonist LP102-6-1 (3.3 g, 30% for three steps). MS m/z 1065.6 [M+H]⁺.

| Linking unit-Agonist | | | |
|---|---|---|---|
| intermediate | Linking unit | Agonist | Mass spectrometry |
| LP102-6-1 | LN102-6 | Resiquimod | Theoretical molecular weight: 1065.20, |
| | | | measured: [½M + H]⁺ = 533.6, 1065.5 |
| | | | [M + H]⁺ = 1065.5 |
| LP102-8-1 | LN102-8 | Resiquimod | [M + H]⁺ = 1079.6 |

### Example 3 Preparation of Targeting Molecule-Pharmaceutical Conjugates

The linking unit-agonist intermediates were respectively conjugated to an antibody in a site-specific manner by a ligase to form an AIAC. The method for conjugation reaction can be found in WO2015165413A1. The resulting AIACs are as listed in the following table:

| | Linking unit-Agonist | | |
|---|---|---|---|
| AIAC | intermediate | A (targeting molecule) | DAR |
| AC102-6-1-1 | LP102-6-1 | Ab0001-LCCT_{L}-HC | 1.74 |
| AC102-8-1-1 | LP102-8-1 | Ab0001-LCCT_{L}-HC | 1.79 |

### Effect Example 1 Assessment of Antibody Immune Agonist Conjugate In Vitro

### Isolation of Human Peripheral Mononuclear Cells

Human peripheral mononuclear cells were isolated from healthy blood donors using SepMate 50 and Lymphoprep (Stem Cell Technologies). The live cells were counted and the cell concentration was adjusted to 1.25×10⁶/ml in RPMI1640 medium with 10% FBS. Tumor cells were detached by trypsin, and collected. The live cells were counted and the cell concentration was adjusted to 2.5×10⁵/ml in RPMI1640 medium with 10% FBS. 12.5×10⁴ human PBMC and 2.5×10⁴ tumor cells (PBMC:tumor cells=5:1) were added into wells of 96 well plate, then the antibody or conjugate was added at indicated concentrations. The cell mixture was incubated with drugs for 18 hours, then the cell-free supernatant was collected for human TNFα ELISA.

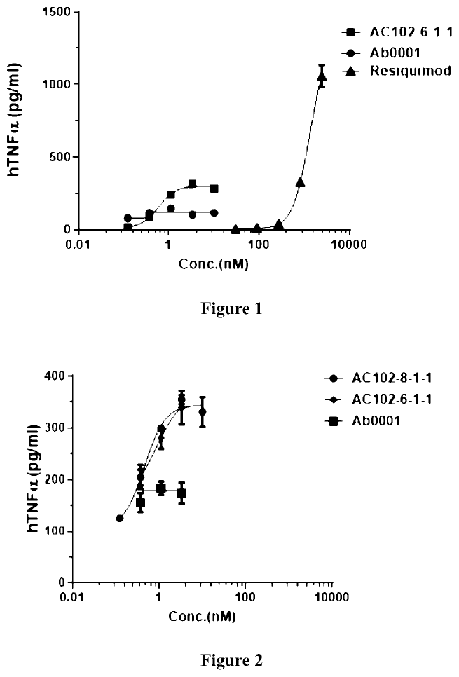

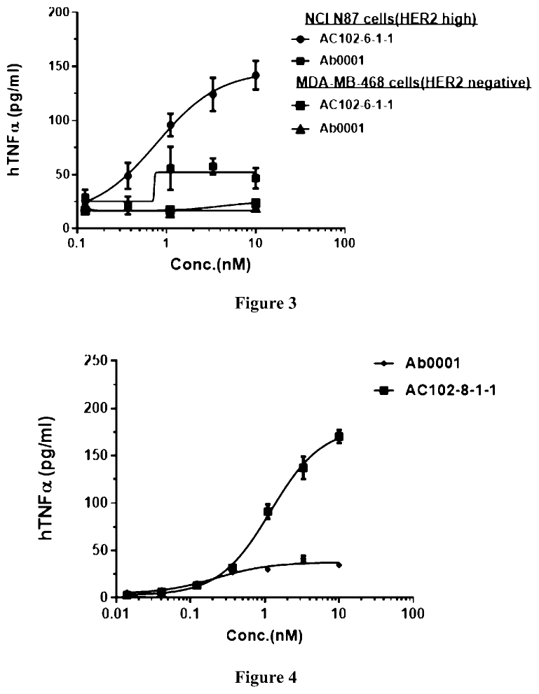

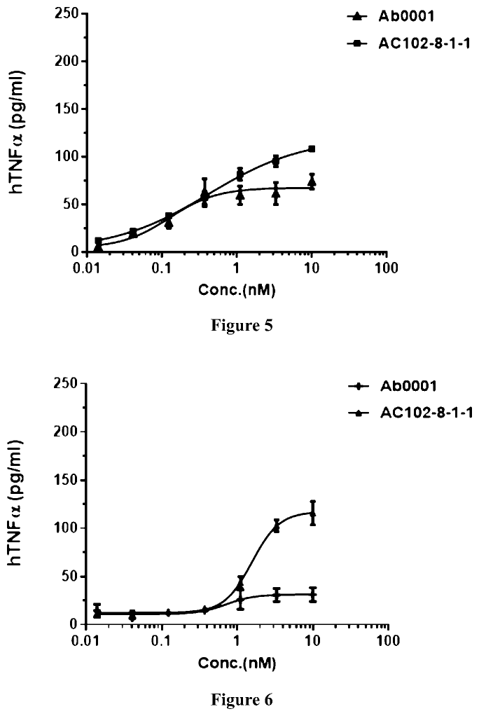

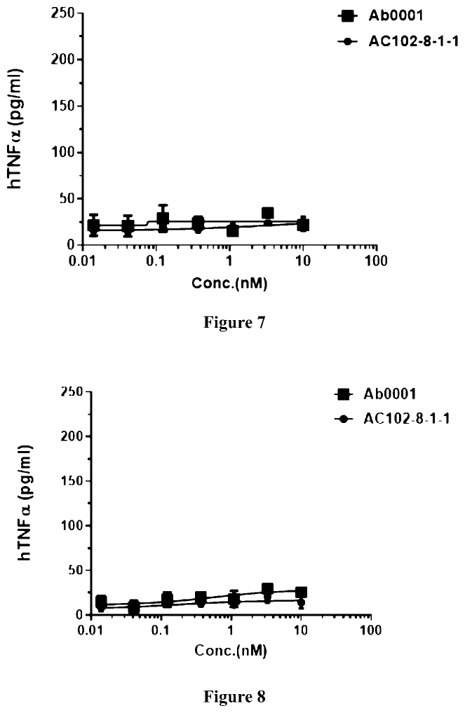

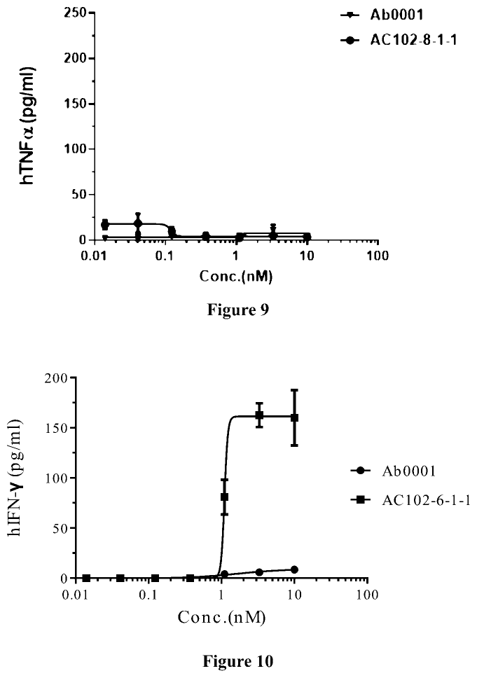

To evaluate the activity of HER2 targeted immunoconjugates, human PBMC and NCI-N87 human gastric cancer cells were co-cultured at a ratio of 5:1, and the antibody or the test immunoconjugate (AC102-6-1-1 or AC102-8-1-1) at indicated concentrations were added. AC102-6-1-1 induced higher TNFα production than the antibody Ab0001, and the effective concentration of AC102-6-1-1 was much lower than the payload Resiquimod (FIG. 1). AC102-8-1-1 induced higher level of TNFα than Ab0001, which was similar to AC102-6-1-1 (FIG. 2). The activity of AC102-6-1-1 was not observed in co-culture of human PBMC and MDA-MB-468 HER2 negative cells, indicating the activity of AC102-6-1-1 was highly dependent on HER2 expression on target tumor cells (FIG. 3). In light of this data, the activity of immunoconjugate was tested in co-culture of human PBMC and other cancer cells with different HER2 expression level, including HCC1954 (FIG. 4), SK-BR-3 (FIG. 5), BT474 (FIG. 6), JIMT1 (FIG. 7), Colo205 (FIG. 8), MDA-MB-468 (FIG. 9). The data showed that AC102-8-1-1 was only capable to induce TNFα in co-culture of PBMC and HER2 high tumor cells.

### Effect Example 2 Assessment of Antibody Immune Agonist Conjugate In Vitro

To evaluate the activity of HER2 targeting immunoconjugates, human PBMC and SK-BR-3 (FIG. 10) or HCC1954 (FIG. 11) human breast cancer cells were co-cultured at a ratio of 5:1, and immunoconjugate (AC102-6-1-1) and antibody (Ab0001) at indicated concentrations were added. Cells were incubated drugs for 18 hours, then cell-free supernatant were collected for human IFNγ detection by ELISA. The isolation of human PBMC and experimental setting were similar to Effect Example 1. AC102-6-1-1 induced higher IFNγ production than the antibody Ab0001, suggesting potential capability to activate T cell response.

### Effect Example 3 Assessment of Antibody Immune Agonist Conjugate In Vivo

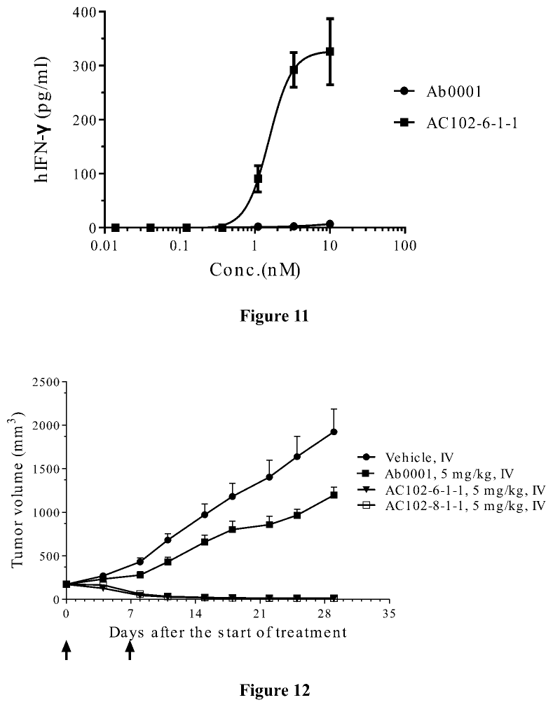

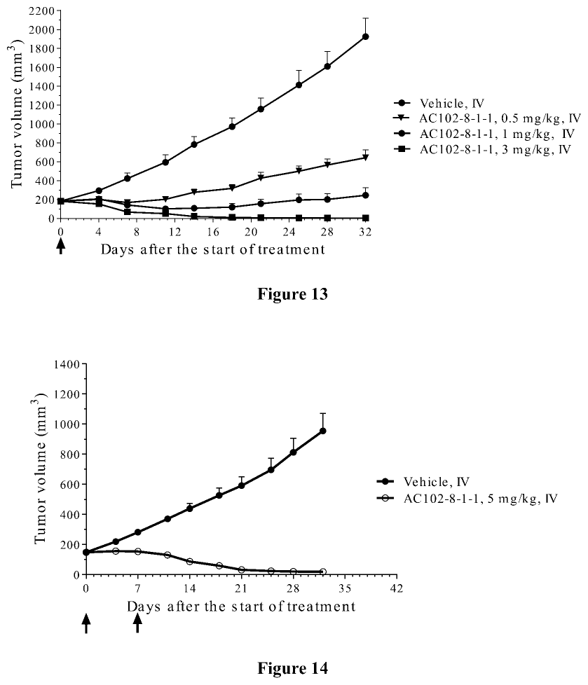

For in vivo anti-tumor efficacy study, 1×10⁷ NCI-N87 human gastric cancer cells were inoculated subcutaneously in the right flank of SCID Beige mice. After 6 days, when tumor volume reached 173 mm³ on average, the tumor bearing mice were assigned and administered intravenously of Ab0001 or the test immunoconjugate (AC102-6-1-1 or AC102-8-1-1) at 5 mg/kg. The tumor volume was measured twice weekly with a caliper. The antibody itself, Ab0001, showed very limited anti-tumor activity. AC102-6-1-1 and AC102-8-1-1 almost cured the tumors at the end (FIG. 12). In a separate study, AC102-8-1-1 displayed dose-dependent activity at 0.5, 1, and 3 mg/kg (FIG. 13).

5×10⁶ JIMT1 human breast cancer cells were inoculated subcutaneously in the right flank of SCID Beige mice to generate xenograft model. After 9 days, when tumor volume reached 149 mm³ on average, the tumor bearing mice were administered intravenously of AC102-8-1-1 at 5 mg/kg. The tumor growth was significantly inhibited (FIG. 14).

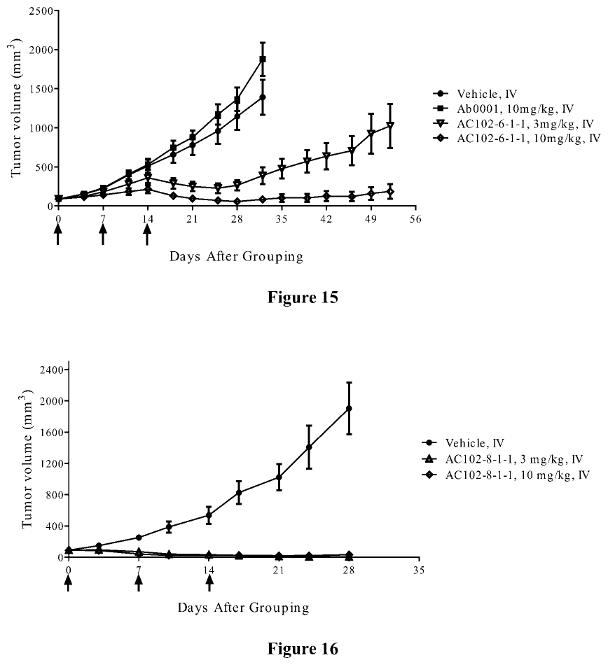

5×10⁵ MC38 hHER2 murine colorectal cancer cells overexpressing human HER2 were inoculated subcutaneously in the right flank of C57BL/6 mice. After 8 days, when tumor volume reached 90 mm³ on average, the tumor bearing mice were assigned and administered intravenously of Ab0001 or AC102-6-1-1. 10 mg/kg Ab0001 showed no obvious anti-tumor activity. 3 mg/kg and 10 mg/kg AC102-6-1-1 dose-dependently inhibited tumor growth (FIG. 15). In a similar setting, AC102-8-1-1 at 3 mg/kg and 10 mg/kg both induced complete tumor regression in 100% of mice (FIG. 16).

### Example of the Formulation Comprising an AIAC

### 1. Example of the pH

### I. Preliminary Selection of the Stable pH Range of Formulation.

### I.1 Experimental Materials

Sample: AC102-8-1-1; Batch NO. 20200815;

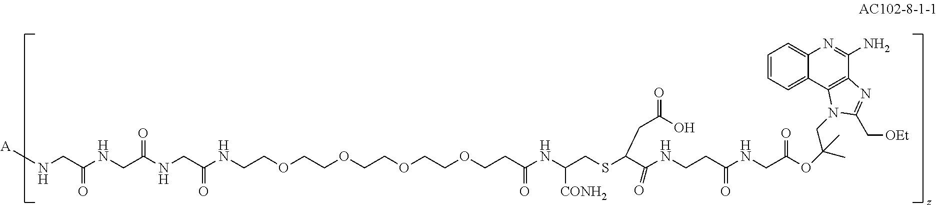

z is an integer of 1 to 4;
A is modified antibody Trastuzumab.

### I.2 Experimental Method

Prepared 5 groups of formulation buffers with different pH (pH values were 5.0, 6.0, 7.0, 8.0, 9.0), and the 5 groups of formulation buffers were as follows:

| | Formula | | | |
|---|---|---|---|---|
| Number | ID | Buffer | Excipient | pH |
| 1 | C5Su | 20 mM Sodium Citrate | 9% (w/v) sucrose | 5.0 |
| 2 | H6Su | 20 mM L-Histidine | 9% (w/v) sucrose | 6.0 |
| 3 | P7Su | 20 mM Sodium Phosphate | 9% (w/v) sucrose | 7.0 |
| 4 | T8Su | 20 mM Tris | 9% (w/v) sucrose | 8.0 |
| 5 | G9Su | 20 mM Glycine | 9% (w/v) sucrose | 9.0 |

The AC102-8-1-1 samples were buffer exchanged using the 5 groups of buffers by dialysis, and the protein concentration was adjusted to about 20 mg/ml. By examining the stability of AC102-8-1-1 at 2-8° C., 25° C. and 40° C. in different pH Buffers, the stable pH range was determined.

The sample preparation procedure was as follows:
a) Thawing samples.
b) Loading sample into dialysis tubing.
c) Dialyzing against 100-fold sample volume buffer for 2 h at room temperature.
d) Changing the dialysis buffer and dialyzing overnight at 2-8° C., twice.
e) Testing protein concentration and adjusting to 20 mg/ml.
f) Sterile-filtering samples by using 0.22 μm filter.
g) Filling, labeling samples, and testing stability.

The stability test conditions and time were shown in the following table:

| | Time | | | | | | | | |
|---|---|---|---|---|---|---|---|---|---|
| | | 1 | 3 | 5 | 7 | 2 | 4 | 8 | 12 |
| Storage Condition | T0 | day | days | days | days | weeks | weeks | weeks | weeks |
| 2-8° C. | x | | | | | | | | x |
| 25° C. | | | | | | x | x | x | x |
| 40° C. | | x | x | x | x | | | | |

Test items: purity (reduced CE-SDS method, non-reduced CE-SDS method), DAR value (HIC-HPLC), free drug (RP-HPLC).

### Detection Method

### Purity (Reduced CE-SDS)

The test sample was diluted with ultrapure water to about 10 mg protein per 1 mL as the test solutions. 85 μL of sample buffer (4 mL pH 6.2 citric acid-phosphate buffer plus 1 mL 10% SDS, water was added to make up to 25 mL) and 5 μL of B-mercaptoethanol were added into 10 μL of the test solution. The solution was vortexed and mixed well. Then the test solution was heated at 70° C. for 5 minutes, cooled to room temperature, centrifuged at 13,000 rpm for 10 minutes before loading, and 85 μL of the supernatant was injected into sample vial for analysis. The loading voltage was 5 KV, the loading time was 20 seconds, the separation voltage was 15 KV, the separation time was 35 minutes, and the detector wavelength was 220 nm. The sum of the percentages of the corrected peak areas of the light chain and the heavy chain was the sample purity.

### Purity (Non-Reduced CE-SDS)

The test sample was diluted to about 10 mg/ml with ultrapure water as the test solution. 85 μL of sample buffer (4 mL pH 6.2 citric acid-phosphate buffer plus 1 mL 10% SDS, water was added to make up to 25 mL) and 5 μL of 500 mM iodoacetamide were added into 10 μL of the test solution. The solution was vortexed and mixed well. Then the test solution was heated at 70° C. for 5 minutes, cooled to room temperature, centrifuged at 13,000 rpm for 10 minutes before loading, and the supernatant was injected into sample vial for analysis. The loading voltage was 5 KV, the loading time was 20 seconds, the separation voltage was 15 KV, the separation time was 35 minutes, and the detector wavelength was 220 nm. The percentage of the corrected peak area of the monomer peak was the sample purity.

### DAR Value (HIC)

DAR value analysis was performed by hydrophobic chromatography. The analytical column was TSK gel Butyl NPR (4.6 mm×10 cm, particle size 2.5 μm); 25 mmol/L phosphate buffer+2 mol/L ammonium sulfate solution (pH 7.0) was used as mobile phase A. Mobile phase B consisted of 25 mmol/L phosphate solution (pH 7.0) mixed with isopropanol at a volume ratio of 70:30. The flow rate was 0.3 mL per minute, the column temperature was 30° C., and the detection wavelength was 280 nm. The test sample was diluted with purified water to a solution containing about 5 mg per 1 mL, as the test solution. Injection volume was 5 μL. Elution gradient was performed according to the table below.

| Time (minute) | Phase A(%) | Phase B(%) |
|---|---|---|
| 0.00 | 90.0 | 10.0 |
| 5.00 | 75.0 | 25.0 |
| 35.00 | 55.0 | 45.0 |
| 35.10 | 90.0 | 10.0 |
| 45.00 | 90.0 | 10.0 |

The area percentages of DAR0, DAR1 and DAR2 were calculated according to the area normalization method, DAR=DAR1%+DAR2%*2.

Free drug (RP-HPLC)

Free drug detection was performed by reversed-phase high performance liquid chromatography. The analytical column was ACQUITY PREMIER C18AX, 2.1 mm×150 mm, and particle size was 1.7 μm·10 mmol/L ammonium acetate aqueous solution was used as mobile phase A, and acetonitrile solution was used as mobile phase B. The flow rate was 0.3 mL per minute, the column temperature was 40° C., and the detection wavelength was 248 nm. 400 μL of acetone was added into 200 μL of the test sample, and the solution was mixed well. The solution was centrifuged at 13,000 rpm for 15 minutes, and the supernatant was taken as the test solution. An appropriate amount of reference substance was prepared into Linker-Agonist reference substance solution containing 0.267 μg-5.3 μg per 1 mL and Agonist reference substance solution containing 0.067 μg-1.3 μg per 1 mL. Injection volume was 3 μl. And the reference solution was operated in the same way. Elution gradient was performed according to the table below.

| Time (minute) | Phase A(%) | Phase B(%) |
|---|---|---|
| 0.0 | 95.0 | 5.0 |
| 14.0 | 50.0 | 50.0 |
| 14.5 | 5.0 | 95.0 |
| 15.5 | 5.0 | 95.0 |
| 16.0 | 95.0 | 5.0 |
| 20.0 | 95.0 | 5.0 |

The content of free drug was calculated by external standard method.

### I.3 Results

The results of the purity test were shown as follows:

The results of non-reduced CE-SDS purity (%) test:

| | Condition | | | | | | | | | |
|---|---|---|---|---|---|---|---|---|---|---|
| | | 40° C. | | | | 25° C. | | | | 2-8° C. |
| Formula | T0 | 1 d | 3 d | 5 d | 7 d | 2 W | 4 W | 8 W | 12 W | 12 W |
| C5Su | 95.8 | 95.7 | 95.6 | 95.2 | 94.8 | 96.0 | 95.0 | 94.9 | 94.7 | 96.2 |
| H6Su | 95.5 | 95.5 | 95.6 | 95.0 | 94.7 | 96.0 | 95.2 | 94.4 | 93.4 | 96.3 |
| P7Su | 95.4 | 94.9 | 94.6 | 94.1 | 93.2 | 96.1 | 95.5 | 93.9 | 91.9 | 96.3 |
| T8Su | 95.3 | 93.7 | 92.6 | 91.1 | 83.9 | 94.6 | 91.2 | N/A | N/A | 95.0 |
| G9Su | 95.6 | 93.3 | 88.9 | 87.1 | 82.1 | 93.3 | 87.4 | N/A | N/A | 95.4 |

Note:
“N/A” indicates the figure was not detected.

The results of reduced CE-SDS purity (%) test:

| | Condition | | | | | | | | | |
|---|---|---|---|---|---|---|---|---|---|---|
| | | 40° C. | | | | 25° C. | | | | 2-8° C. |
| Formula | T0 | 1 d | 3 d | 5 d | 7 d | 2W | 4 W | 8 W | 12 W | 12 W |
| C5Su | 97.8 | 97.7 | 97.7 | 97.2 | 96.2 | 97.3 | 96.5 | 96.2 | 96.0 | 97.5 |
| H6Su | 97.5 | 97.5 | 97.1 | 96.5 | 96.1 | 96.7 | 96.2 | 95.1 | 92.8 | 97.5 |
| P7Su | 97.6 | 97.0 | 93.2 | 91.5 | 89.9 | 94.3 | 93.2 | 89.4 | 87.8 | 97.2 |
| T8Su | 97.6 | 93.9 | 88.9 | 84.5 | 77.2 | 89.3 | 82.3 | N/A | N/A | 91.4 |
| G9Su | 94.8 | 87.7 | 77.6 | 67.5 | 57.7 | 78.4 | 65.0 | N/A | N/A | 83.0 |

The results of DAR value detection:

| | Condition | | | | | | | | | |
|---|---|---|---|---|---|---|---|---|---|---|
| | | 40° C. | | | | 25° C. | | | | 2-8° C. |
| Formula | T0 | 1 d | 3 d | 5 d | 7 d | 2 W | 4 W | 8 W | 12 W | 12 W |
| C5Su | 1.88 | 1.88 | N/A | 1.87 | 1.86 | 1.89 | 1.88 | 1.88 | 1.80 | 1.85 |
| H6Su | 1.88 | 1.87 | N/A | 1.84 | 1.83 | 1.86 | 1.87 | 1.82 | 1.75 | 1.84 |
| P7Su | 1.87 | 1.85 | N/A | 1.81 | 1.79 | 1.84 | 1.82 | 1.75 | 1.59 | 1.82 |
| T8Su | 1.86 | 1.80 | N/A | 1.64 | 1.56 | 1.70 | 1.45 | N/A | N/A | 1.66 |
| G9Su | 1.85 | 1.69 | N/A | 1.25 | 1.05 | 1.31 | 0.90 | N/A | N/A | 1.34 |

The results of free drug detection:

| | | 25° C. | 25° C. | 25° C. | 2-8° C. |
|---|---|---|---|---|---|
| | Formula | 4 W | 8 W | 12 W | 12 W |
| Residual | C5Su | 34 | 40 | 36 | 12 |
| linker- | H6Su | 44 | 46 | 40 | 20 |
| agonist | P7Su | 62 | 57 | 45 | 17 |
| (ppm) | T8Su | 76 | N/A | N/A | 19 |
| | G9Su | 54 | N/A | N/A | 25 |
| Residual | C5Su | 11 | 65 | 99 | 3 |
| Agonist | H6Su | 40 | 169 | 199 | 15 |
| (ppm) | P7Su | 163 | 368 | 442 | 32 |
| | T8Su | 824 | N/A | N/A | 377 |
| | G9Su | 1984 | N/A | N/A | 1016 |

### I.4 Conclusion

The analysis results of the reduced CE-SDS purity analysis method, the non-reduced CE-SDS purity analysis method, the DAR value detection (HIC-HPLC), and the free drug (RP-HPLC) analysis show that: at pH 7.0, 8.0, 9.0, the molecular stability of AIAC is poor; the protein is more likely to be degraded; the small molecules conjugated with the antibody are more likely to fall off; and the DAR value decreases more significantly. Therefore, the AIAC is not suitable for long-term storage at pH 7.0-9.0.

### II. The Optimal pH Range of the AIAC was Further Screened in the Range of pH 4.0-pH 6.0.

### II.1 Experimental Materials

### Sample: AC102-8-1-1; Batch NO. 20200815.

### II.2 Experimental Method

Prepared 4 groups of formulation buffers with different pH (pH values are 4.0, 5.0, 5.5, 6.0 respectively), and the 4 groups of formulation buffers were as follows:

| | Formula | | | |
|---|---|---|---|---|
| Number | ID | Buffer | Excipient | pH |
| 1 | A4Su | 20 mM Sodium Acetate | 9% (w/v) sucrose | 4.0 |
| 2 | C5Su | 20 mM Sodium Citrate | 9% (w/v) sucrose | 5.0 |
| 3 | H5.5Tr | 20 mM L-Histidine | 9% (w/v) trehalose | 5.5 |
| | | | dihydrate | |
| 4 | H6Su | 20 mM L-Histidine | 9% (w/v) sucrose | 6.0 |

The AC102-8-1-1 samples were buffer exchanged using the 4 groups of buffers by dialysis, and the protein concentration was adjusted to about 20 mg/ml. By examining the stability of AC102-8-1-1 at 2-8° C., 25° C. and 40° C. in different pH Buffers, the stable pH range was determined. The sample preparation procedure was the same as Example 1.2:

The stability test conditions and time were shown in the following table:

| | Time Points | | | | | | | | |
|---|---|---|---|---|---|---|---|---|---|
| | | 1 | 3 | 5 | 7 | 2 | 4 | 8 | 12 |
| Storage Conditions | T0 | day | days | days | days | weeks | weeks | weeks | weeks |
| 2-8° C. | x | | | | | | | | x |
| 25° C. | | | | | | x | x | x | x |
| 40° C. | | x | x | x | x | | | | |

Test items: purity (SE-HPLC), charge variants (CEX-HPLC), Tₘ/T_{agg}.

### Detection Method

### Purity (SE-HPLC)

Sample purity was checked by size exclusion high performance liquid chromatography (SE-HPLC). The analytical column was TSKgel G3000SWXL, 7.8×300 mm; the mobile phase was 0.2 mol/L potassium dihydrogen phosphate-0.25 mol/L potassium chloride solution, pH was 6.2; the test sample was diluted with ultrapure water to about 2 mg per 1 mL as the test solution, and 50 μL of the test solution was injected into the high performance liquid chromatograph. The column temperature was 25° C., the flow rate was 0.5 mL per minute, the detection wavelength was 280 nm, and the elution time was 30 minutes. The percentages of aggregate, monomer and fragment components were calculated using area normalization.

### Charge Variants (CEX-HPLC)

Charge heterogeneity was detected by cation exchange high performance liquid chromatography (CEX-HPLC). The column was a weak cation exchange column (Propac WCX-10, 4×250 mm); 10 mM phosphate buffer (5 mM NaH₂PO₄·2H₂O+5 mM Na₂HPO₄·12H₂O) was used as Phase A, 10 mM phosphate buffer (5 mM NaH₂PO₄·2H₂O+5 mM Na₂HPO₄·12H2O) and 200 mM sodium chloride were used as Phase B, flow rate was 1.0 mL per minute, detection wavelength was 280 nm. The test sample was diluted with ultrapure water to about 2 mg per 1 mL as the test solution. 50 μL of the test solution was injected into the liquid chromatograph, and the elution gradient was performed according to the table below.

| Time (minute) | Phase A(%) | Phase B(%) |
|---|---|---|
| 0.0 | 100 | 0 |
| 30.0 | 50 | 50 |
| 31.0 | 0 | 100 |
| 35.0 | 0 | 100 |
| 35.1 | 100 | 0 |
| 45.0 | 100 | 0 |

The percentages of main components, acidic components, and basic components were reported by calculating according to the area normalization method.

$${T}_{m} / {T}_{agg}$$

Tₘ and T_{agg} analysis were performed using the Uncle analysis system (Unchained Labs). The analysis parameters were as follows:
SLS Settings: Exp: Exp. 1; Start Temp (° C.): 25; Incubation Time (sec): 180; Ramp Rate (° C./min): 0.7; Plate Hold (sec): 45; End Temp (° C.): 95.
DLS Settings: Initial Acquisition: Yes; Final Acquisition: Yes; No. of Acquisitions: 4; Acquisition Time (sec): 5; Attenuator Control: Auto; Laser Control: Auto.

### II.3 Results

The results of purity (%) (SEC-HPLC) test:

| | | Condition | | | | |
|---|---|---|---|---|---|---|
| | | 40° C. | | | | |
| Formula | Components | 0 d | 1 d | 3 d | 5 d | 7 d |
| A4Su | Monomer | 95.4 | 95.4 | 95.4 | 94.5 | 94.5 |
| | Aggregate | 4.6 | 4.6 | 4.6 | 4.5 | 4.3 |
| | Fragment | 0.0 | 0.0 | 0.0 | 1.0 | 1.2 |
| C5Su | Monomer | 95.3 | 95.3 | 95.3 | 95.4 | 94.4 |
| | Aggregate | 4.7 | 4.7 | 4.7 | 4.6 | 4.7 |
| | Fragment | 0.0 | 0.0 | 0.0 | 0.0 | 0.9 |
| H5.5Tr | Monomer | 95.4 | 95.3 | 95.4 | 95.3 | 95.4 |
| | Aggregate | 4.6 | 4.7 | 4.6 | 4.7 | 4.7 |
| | Fragment | 0.0 | 0.0 | 0.0 | 0.1 | 0.0 |
| H6Su | Monomer | 95.3 | 95.3 | 95.3 | 95.3 | 95.3 |
| | Aggregate | 4.7 | 4.7 | 4.7 | 4.6 | 4.7 |
| | Fragment | 0.0 | 0.0 | 0.0 | 0.0 | 0.0 |

The results of charge variants (%) (CEX-HPLC) analysis:

| Condition | | 25° C. | | | | |
|---|---|---|---|---|---|---|
| Formula | Components | 0 W | 2 W | 4 W | 8 W | 12 W |
| A4Su | Acidic | 36.9 | 30.0 | 29.5 | 27.0 | 28.9 |
| | Main | 51.6 | 44.4 | 35.6 | 27.7 | 24.9 |
| | Basic | 11.5 | 25.7 | 35.0 | 45.3 | 46.2 |
| C5Su | Acidic | 37.3 | 33.5 | 34.0 | 29.7 | 28.7 |
| | Main | 50.4 | 42.2 | 33.9 | 26.7 | 23.0 |
| | Basic | 11.9 | 24.3 | 32.1 | 43.5 | 48.3 |
| H5.5Tr | Acidic | 38.1 | 33.0 | 37.1 | 43.3 | 40.5 |
| | Main | 50.5 | 48.1 | 38.8 | 29.0 | 26.3 |
| | Basic | 11.4 | 18.9 | 23.9 | 27.6 | 33.3 |
| H6Su | Acidic | 38.5 | 40.2 | 45.0 | 52.4 | 53.1 |
| | Main | 50.2 | 43.9 | 35.5 | 26.8 | 23.7 |
| | Basic | 11.3 | 15.9 | 19.4 | 20.7 | 23.1 |

The results of protein thermal stability analysis:

| | |
|---|---|
| Tm/° C. | T_{agg}/° C. |

| Formula | Tm1 | Tm2 | T_{agg} 266 | T_{agg} 473 |
|---|---|---|---|---|
| A4Su | 62.68 | 79.24 | 31.46 | 48.82 |
| C5Su | ND | 80.82 | 78.29 | 78.89 |
| H5.5Tr | 68.64 | 86.10 | 81.11 | 82.69 |
| H6Su | 70.74 | 84.55 | 81.58 | 82.22 |

Note:
“ND” indicates the figure was not detected; T_{agg}266 refers to measuring SLS (static light scattering) at 266 nm, which is more sensitive and suitable to detect smaller particles; T_{agg}473 refers to measuring SLS at 473 nm, which is more sensitive and suitable to detect larger particles; Tm1 is associated with C_{H}2 dissociation of antibody and Tm2 is associated with C_{H}3 dissociation of antibody.

### II.4 Conclusion

At pH 4.0 and 5.0, more fragments were produced, and the purity decreased more rapidly. The results of protein thermal stability analysis also showed 10 that the AIAC molecules had lower Tm and Tagg values at low pH, and were easier to aggregation. When the pH is 6.0, the acid component rose faster. Therefore, the optimum pH was finally screened to be 5.5.

### 2. The Screening of Filler

### 2.1 the Purpose of the Experiment

Screened out fillers (stabilizers) to stabilize AIAC.

### 2.2 Experimental Materials

### Sample: AC102-8-1-1; Batch NO. 20200815.

0.3 Experimental Method

Protein stabilizers commonly used for biological products include sucrose, trehalose, and sorbitol. In this example, 3 groups of different formulation prescriptions were prepared (the pH values of the 3 groups of prescriptions were all set to 5.5, and the 3 groups of prescriptions contained 9% sucrose, 9% trehalose, and 5% sorbitol respectively), and the 3 groups of formulation prescriptions were as follows:

| | | | Protein |
|---|---|---|---|
| NO. | Buffer | Excipient | Concentration |
| 1 | 20 mM L-His pH 5.5 | 9% (w/v) sucrose | 20 mg/mL |
| 2 | 20 mM L-His pH 5.5 | 9%(w/v) trehalose | 20 mg/mL |
| | | dihydrate | |
| 3 | 20 mM L-His pH 5.5 | 5% (w/v) sorbitol | 20 mg/mL |

The AC102-8-1-1 samples were buffer exchanged using the 3 groups of buffers by dialysis, and the protein concentration was adjusted to about 20 mg/ml. By examining the stability of AC102-8-1-1 under different formulations, a suitable stabilizer was screened out. The sample preparation procedure was the same as Example I.2:

### Detection Method

### Binding Activity (ELISA)

Binding activity was determined by enzyme-linked immunosorbent assay (ELISA). The antigen (Human HER2 protein) was added to the microtiter plate, coated overnight at 2-8° C., and blocked with blocking solution. After washing the plate with washing solution, the reference solution and the test solution were added respectively, and incubated at 25° C. for 1 hour; after washing the plate with washing solution, the mouse anti-human IgG-Fc antibody solution labeled with horseradish peroxidase was added to the ELISA plate, and incubated with shaking at 25° C. for 1 hour; after washing the plate with washing solution, TMB was added for color development, and the color was developed in the dark for 10 min; after the reaction was terminated with termination solution, the absorbance was measured at a wavelength of 450 nm. A four-parameter logarithmic regression (4PL) model was used to fit the EC₅₀ of the reference and test articles, respectively. The relative activity of the test article was calculated by reference article EC₅₀/test article EC₅₀.

### 2.4 Results

The results of purity (SEC-HPLC) test (Aggregate %):

| | |
|---|---|
| 40° C. (Week) | Shaking (Day) |

| NO. | T0 | 1 | 2 | 3 | 7 |
|---|---|---|---|---|---|
| 1 | 4.6 | 4.8 | 5.0 | 4.8 | 5.1 |
| 2 | 4.6 | 4.6 | 4.9 | 4.6 | 5.3 |
| 3 | 4.7 | 4.7 | 5.0 | 4.6 | 5.1 |

The results of relative binding activity assay:

| NO. | T0 | 2-8° C. 12 W |
|---|---|---|
| 1 | 100% | 98% |
| 2 | 105% | 97% |
| 3 | 108% | 103% |

### 2.5 Conclusion

Combining SEC and binding activity results, sucrose was superior to trehalose and sorbitol as a filler agent (stabilizer).

### 3. Study on the Effect of Surfactant

### 3.1 the Purpose of the Experiment

The effects of surfactants on the formulation stability of the AIAC were investigated.

### 3.2 Experimental Materials

Sample: AC102-8-1-1; Batch NO. 20200815.

### 3.3 Experimental Method

Prepared groups 1-3 did not contain Tween 20, and groups 4-7 contained 0.2 mg/ml Tween 20. The Buffer, pH value and protein concentration of all 7 groups of prescriptions remained the same. The prescriptions of the 7 groups of formulations were as follows:

| | | | Protein |
|---|---|---|---|
| NO. | Buffer | Excipient | Concentration |
| 1 | 20 mM L-His pH 5.5 | 9% (w/v) sucrose | 20 mg/mL |
| 2 | 20 mM L-His pH 5.5 | 9% (w/v) trehalose dihydrate | 20 mg/mL |
| 3 | 20 mM L-His pH 5.5 | 5% (w/v) sorbitol | 20 mg/mL |
| 4 | 20 mM L-His pH 5.5 | 4.5% (w/v) sucrose + 150 mM | 20 mg/mL |
| | | Lysine HCl + 0.2 mg/mL PS20 | |
| 5 | 20 mM L-His pH 5.5 | 4.5% (w/v) sucrose + 150 mM | 20 mg/mL |
| | | Arginine HCl + 0.2 mg/mL PS20 | |
| 6 | 20 mM L-His pH 5.5 | 4.5% (w/v) sucrose + 150 mM | 20 mg/mL |
| | | Arginine HCl + 1.49 mg/ml | |
| | | Met + 0.2 mg/mL PS20 | |
| 7 | 20 mM L-His pH 5.5 | 9% (w/v) sucrose + 1.49 mg/ml | 20 mg/mL |
| | | Met + 0.2 mg/mL PS20 | |

Note:
“Met” indicates Methionine.

The AC102-8-1-1 samples were buffer exchanged using the 7 groups of buffers by dialysis, and the protein concentration was adjusted to about 20 mg/ml. By examining the stability of AC102-8-1-1 under different formulations, the protective effect of surfactant (Tween 20, PS20) on the AIAC protein was determined. The sample preparation procedure was the same as Example 1.2.

Detection parameters: visible foreign matter inspection (visual method) (F/T is the abbreviation of Freeze/Thawing).

### 3.4 Results

| | 2-8° C. | | −40° C. | | 25° C. | | | | 40° C. | | Shaking | | F/T | |
|---|---|---|---|---|---|---|---|---|---|---|---|---|---|---|
| | (W) | | (W) | | (W) | | | | (W) | | (D) | | (Cycles) | |
| NO. | 0 | 12 | 4 | 12 | 2 | 4 | 8 | 12 | 1 | 2 | 3 | 7 | 2 | 5 |
| 1 | ✓ | ✓ | 2 | ✓ | ✓ | ✓ | ✓ | ✓ | ✓ | ✓ | ✓ | ✓ | 1 | 1 |
| 2 | ✓ | ✓ | 2 | ✓ | ✓ | ✓ | ✓ | ✓ | ✓ | ✓ | ✓ | ✓ | 1 | 1 |
| 3 | ✓ | ✓ | 2 | ✓ | ✓ | ✓ | ✓ | 2 | ✓ | ✓ | ✓ | ✓ | 1 | 1 |
| 4 | ✓ | ✓ | ✓ | ✓ | ✓ | ✓ | ✓ | ✓ | ✓ | ✓ | ✓ | ✓ | ✓ | ✓ |
| 5 | ✓ | 3 | ✓ | ✓ | ✓ | ✓ | ✓ | ✓ | ✓ | ✓ | ✓ | ✓ | ✓ | ✓ |
| 6 | ✓ | ✓ | 3 | ✓ | ✓ | ✓ | ✓ | ✓ | ✓ | ✓ | ✓ | ✓ | ✓ | ✓ |
| 7 | ✓ | ✓ | ✓ | ✓ | ✓ | ✓ | ✓ | ✓ | ✓ | ✓ | ✓ | ✓ | ✓ | ✓ |

✓: Colorless clear liquid. No visible particle.
1: White precipitation was detected.
2: There are small visible particles.
3: 1 white fiber was detected (Extrinsic particulates).

### 3.5 Conclusion

Tween 20 could effectively inhibit the aggregation and precipitation of the AIAC molecules during freeze-thawing and freezing.

Combined with all the previous screening results, the formulation of the AIAC was determined to be:
20 mM His, pH 5.5 (Buffer)
9% (W/V) sucrose (Stabilizer)
0.2 mg/mL PS 20 (Stabilizer and surfactant).

### 4. Formulation Concentration of the AIAC

### 4.1 the Purpose of the Experiment

Further selection of suitable protein concentration range after formulation composition was determined.

### 4.2 Experimental Materials

Sample: AC102-6-1-1; Batch NO. 20210114;

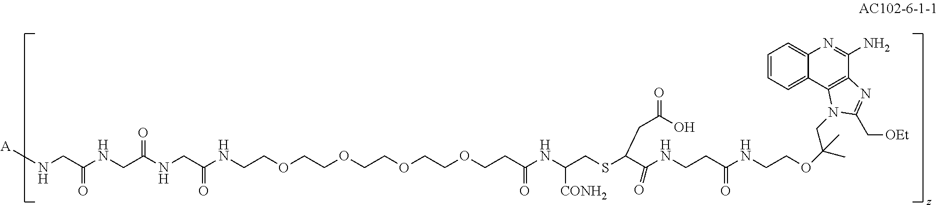

z is an integer of 1 to 4;
A is modified antibody Trastuzumab.

### 4.3 Experimental Method

As shown in the table below, the protein concentrations of groups 1 to 3 were 20 mg/mL, 40 mg/mL, and 60 mg/mL, respectively. Other formulation components were the same. The prescriptions of the 3 groups of formulations were as follows:

| | | | | Protein |
|---|---|---|---|---|
| NO. | Buffer | Excipient 1 | Excipient 2 | Concentration |
| 1 | 20 mM L-His pH 5.5 | 9% (w/v) sucrose | 0.2 mg/mL PS20 | 20 mg/mL |
| 2 | 20 mM L-His pH 5.5 | 9% (w/v) sucrose | 0.2 mg/mL PS20 | 40 mg/mL |
| 3 | 20 mM L-His pH 5.5 | 5% (w/v) sucrose | 0.4 mg/mL PS20 | 60 mg/mL |

The AC102-6-1-1 samples were buffer exchanged using the 3 groups of buffers by dialysis, and the protein concentration was adjusted to about 20 mg/ml, 40 mg/mL, and 60 mg/mL, respectively. By examining the stability of AC102-6-1-1 at different protein concentrations, the appropriate protein concentration range was determined. The sample preparation procedure was the same as Example 1.2.

The inspection conditions and sampling time points were as follows (F/T is the abbreviation of Freeze/Thawing):

| Storage | | | 2 | 5 | 3 | 1 | 2 | 4 | 8 | 12 |
|---|---|---|---|---|---|---|---|---|---|---|
| Conditions | | T0 | cycles | cycles | days | week | weeks | weeks | weeks | weeks |
| −20° | C. | | | | | | | x | | x |
| 2-8° | C. | x | | | | | | | | x |
| 25° | C. | | | | | | x | x | x | x |
| Shaking | | | | | x | x | | | | |
| F/T | | | x | x | | | | | | |
| −40° | C. | | | | | | | | | x |
| −80° | C. | | | | | | | | | x |

Detection parameters: visible particle inspection (visual method), SEC-HPLC purity detection, non-reduced CE-SDS purity detection, reduced CE-SDS purity detection, DAR value detection, free drug detection. The testing methods are similar as that above.

### 4.4 Results

The results of visible particle detection:

| | | Shaking | | F/T (times) | | 25° C. | | | | −20° C. | −20° C. | 2-8° C. |
|---|---|---|---|---|---|---|---|---|---|---|---|---|
| NO. | T0 | 3 d | 7 d | 2 | 5 | 2 W | 4 W | 8 W | 12 W | 4 W | 12 W | 12 W |
| 1 | ✓ | ✓ | ✓ | ✓ | ✓ | ✓ | ✓ | ✓ | ✓ | ✓ | ✓ | ✓ |
| 2 | ✓ | ✓ | ✓ | ✓ | ✓ | ✓ | ✓ | ✓ | ✓ | ✓ | ✓ | ✓ |
| 3 | ✓ | ✓ | ✓ | ✓ | ✓ | ✓ | ✓ | ✓ | ✓ | ✓ | ✓ | ✓ |

Note:
✓: Essentially free of visible particle.

The results of SEC-HPLC aggregate percentage (%) detection:

| | | |
|---|---|---|
| −20° C. | Shaking | Freeze/Thawing |
| (Week) | (Day) | (Cycles) |

| NO. | T0 | 4 | 12 | 3 | 7 | 2 | 5 |
|---|---|---|---|---|---|---|---|
| 1 | 3.2 | 3.1 | 2.9 | 3.2 | 3.2 | 3.2 | 3.2 |
| 2 | 3.2 | 3.0 | 2.9 | 3.1 | 3.1 | 2.8 | 3.0 |
| 3 | 3.2 | 3.1 | 2.9 | 3.4 | 3.5 | 3.3 | 3.2 |

| | | 25° C. (Week) | | | | −40° C. | −80° C. | 2-8° C. |
|---|---|---|---|---|---|---|---|---|
| NO. | T0 | 2 | 4 | 8 | 12 | 12 W | 12 W | 12 W |
| 1 | 3.2 | 3.0 | 3.0 | / | 2.9 | 2.9 | 2.9 | 2.9 |
| 2 | 3.2 | 3.0 | 3.1 | / | 3.3 | 2.9 | 2.9 | 3.0 |
| 3 | 3.2 | 3.3 | 3.3 | / | 3.5 | 3.0 | 2.9 | 3.1 |

Note:
“/” indicates that figure was not detected.

The results of non-reduced CE-SDS purity (%) test:

| | | −20° C. | | Shaking | | Freeze/Thawing | |
|---|---|---|---|---|---|---|---|
| | | (Week) | | (Day) | | (Cycles) | |
| NO. | T0 | 4 | 12 | 3 | 7 | 2 | 5 |
| 1 | 95.1 | 96.2 | 95.3 | 94.7 | 94.8 | 95.6 | 95.6 |
| 2 | 94.8 | 95.9 | 95.2 | 94.8 | 95.3 | 95.0 | 95.0 |
| 3 | 95.5 | 95.5 | 95.2 | 95.4 | 95.6 | 95.4 | 95.4 |

| | | 25° C. (Week) | | | | −40° C. | −80° C. | 2-8° C. |
|---|---|---|---|---|---|---|---|---|
| NO. | T0 | 2 | 4 | 8 | 12 | 12 W | 12 W | 12 W |
| 1 | 95.1 | 95.7 | 95.7 | 94.2 | 94.4 | 95.5 | 95.8 | 95.0 |
| 2 | 94.8 | 95.5 | 95.4 | 94.6 | 93.8 | 95.4 | 95.7 | 95.2 |
| 3 | 95.5 | 95.7 | 95.2 | 94.1 | 93.7 | 95.3 | 95.5 | 95.1 |

The results of reduced CE-SDS purity (%) test:

| | | −20° C. | | Shaking | | Freeze/Thawing | |
|---|---|---|---|---|---|---|---|
| | | (Week) | | (Day) | | (Cycles) | |
| NO. | T0 | 4 | 12 | 3 | 7 | 2 | 5 |
| 1 | 97.4 | 97.7 | 97.5 | 97.6 | 97.6 | 98.0 | 97.8 |
| 2 | 97.3 | 97.7 | 97.6 | 97.8 | 97.3 | 97.9 | 97.6 |
| 3 | 97.4 | 97.7 | 97.3 | 97.6 | 97.7 | 97.5 | 97.7 |

| | | 25° C. (Week) | | | | −40° C. | −80° C. | 2-8° C. |
|---|---|---|---|---|---|---|---|---|
| NO. | T0 | 2 | 4 | 8 | 12 | 12 W | 12 W | 12 W |
| 1 | 97.4 | 97.0 | 97.5 | 97.1 | 96.0 | 97.5 | 97.4 | 97.4 |
| 2 | 97.3 | 97.5 | 97.3 | 97.6 | 96.2 | 97.5 | 97.3 | 97.1 |
| 3 | 97.4 | 97.7 | 97.3 | 96.8 | 96.2 | 97.6 | 97.5 | 97.2 |

The results of DAR value test:

| | | −20° C. | | Shaking | | Freeze/Thawing | |
|---|---|---|---|---|---|---|---|
| | | (Week) | | (Day) | | (Cycles) | |
| NO. | T0 | 4 | 12 | 3 | 7 | 2 | 5 |
| 1 | 1.85 | 1.81 | 1.80 | 1.85 | 1.85 | 1.85 | 1.85 |
| 2 | 1.85 | 1.81 | 1.80 | 1.85 | 1.85 | 1.85 | 1.85 |
| 3 | 1.85 | 1.80 | 1.81 | 1.85 | 1.85 | 1.85 | 1.85 |

| | | 25° C. (Week) | | | | −40° C. | −80° C. | 2-8° C. |
|---|---|---|---|---|---|---|---|---|
| NO. | T0 | 2 | 4 | 8 | 12 | 12 W | 12 W | 12 W |
| 1 | 1.85 | 1.81 | 1.81 | 1.80 | 1.78 | 1.82 | 1.82 | 1.81 |
| 2 | 1.85 | 1.82 | 1.81 | 1.80 | 1.78 | 1.82 | 1.81 | 1.80 |
| 3 | 1.85 | 1.80 | 1.81 | 1.80 | 1.78 | 1.81 | 1.81 | 1.80 |

The results of free drug detection (linker-payload, ppm):

| | | |
|---|---|---|
| −20° C. | Shaking | Freeze/Thawing |
| (Week) | (Day) | (Cycles) |

| NO. | T0 | 4 | 12 | 3 | 7 | 2 | 5 |
|---|---|---|---|---|---|---|---|
| 1 | 7.0 | 8.3 | 8.5 | 5.8 | 6.5 | 5.3 | 7.3 |
| 2 | 6.5 | 8.6 | 10.1 | 7.2 | 7.5 | 5.7 | 6.2 |
| 3 | 8.0 | 6.9 | 29.9 | 9.6 | 8.4 | 6.8 | 9.0 |

| | | 25° C. (Week) | | | | −40° C. | −80° C. | 2-8° C. |
|---|---|---|---|---|---|---|---|---|
| NO. | T0 | 2 | 4 | 8 | 12 | 12 W | 12 W | 12 W |
| 1 | 7.0 | 30.5 | 42.0 | / | 149.4 | 4.2 | 5.1 | 33.1 |
| 2 | 6.5 | 33.5 | 44.8 | / | 181.4 | 3.7 | 4.6 | 42.7 |
| 3 | 8.0 | 40.9 | 55.6 | / | 183.3 | 3.8 | 4.6 | 54.3 |

The results of free drug detection (agonist, ppm):

| | | |
|---|---|---|
| −20° C. | Shaking | Freeze/Thawing |
| (Week) | (Day) | (Cycles) |

| NO. | T0 | 4 | 12 | 3 | 7 | 2 | 5 |
|---|---|---|---|---|---|---|---|
| 1 | <2 | <2 | <2 | <2 | <2 | <2 | <2 |
| 2 | <2 | <2 | <2 | <2 | <2 | <2 | <2 |
| 3 | <2 | <2 | <2 | <2 | <2 | <2 | <2 |

| | | 25° C. (Week) | | | | −40° C. | −80° C. | 2-8° C. |
|---|---|---|---|---|---|---|---|---|
| NO. | T0 | 2 | 4 | 8 | 12 | 12 W | 12 W | 12 W |
| 1 | <2 | <2 | <2 | / | <2 | <2 | <2 | <2 |
| 2 | <2 | <2 | <2 | / | <2 | <2 | <2 | <2 |
| 3 | <2 | <2 | <2 | / | <2 | <2 | <2 | <2 |

### 4.5 Conclusion

Visible particle, SEC, reducing and non-reducing CE-SDS detection, DAR value detection, free drug detection results all showed that AC102-6-1-1 had very good stability in the concentration range of 20-60 mg/ml under this formulation.

### 5. High Protein Concentration of the AIAC

### 5.1 the Purpose of the Experiment

Further developing high protein concentration formulations and verifying the stability thereof.

### 5.2 Experimental Materials

### Sample: AC102-6-1-1.

### 5.3 Experimental Method

The prescriptions of the high protein concentration formulations were as follows:

| | | | | Protein |
|---|---|---|---|---|
| NO. | Buffer | Stabilizer | Surfactant | Concentration |
| 1 | 20 mM L-Histidine pH 5.65 | 7.5% (w/v) sucrose | 0.2 mg/ml PS20 | 62.2 mg/ml |
| 2 | 20 mM L-Histidine pH 5.68 | 7.5% (w/v) sucrose | 0.2 mg/ml PS20 | 82.2 mg/ml |
| 3 | 20 mM L-Histidine pH 5.69 | 7.5% (w/v) sucrose | 0.2 mg/ml PS20 | 100.4 mg/ml |
| 4 | 20 mM L-Histidine pH 5.65 | 9% (w/v) sucrose | 0.2 mg/ml PS20 | 62.2 mg/ml |
| 5 | 20 mM L-Histidine pH 5.68 | 9% (w/v) sucrose | 0.2 mg/ml PS20 | 82.2 mg/ml |
| 6 | 20 mM L-Histidine pH 5.69 | 9% (w/v) sucrose | 0.2 mg/ml PS20 | 100.4 mg/ml |

The AC102-6-1-1 samples were buffer exchanged using the 6 groups of buffers by dialysis, and the protein concentration was adjusted to 62.2 mg/ml, 82.2 mg/ml and 100.4 mg/ml, respectively. By examining the stability of AC102-6-1-1 at high protein concentrations, verifying the feasibility of high protein concentration formulations. The sample preparation procedure was the same as Example 1.2.

The inspection conditions and sampling time points were as follows (F/T is the abbreviation of Freeze/Thawing):

| Storage | | F/T 5 | | |
|---|---|---|---|---|
| Conditions | 0 | Cycles | 1 W | 2 W |
| 40° C. | x | | x | x |
| Shaking | | | x | |
| Freeze/thawing | | x | | |

Detection parameters: visible particle inspection (visual method), SEC-HPLC purity detection, non-reduced CE-SDS purity detection.

### 5.4 Results

The results of visible particle detection:

| | | | | | | | T 40° C. | | | |
|---|---|---|---|---|---|---|---|---|---|---|
| Formula | T0 | | Shaking 7 d | | F/T 5 Cycles | | 1 W | | 2 W | |
| ID | Color | Clarity | Color | Clarity | Color | Clarity | Color | Clarity | Color | Clarity |
| 1 | SY | CL | SY | CL | SY | CL | SY | CL | SY | CL |
| 2 | SY | CL | SY | CL | SY | CL | SY | CL | SY | CL |
| 3 | SY | CL | SY | CL | SY | CL | SY | CL | SY | CL |
| 4 | SY | CL | SY | CL | SY | CL | SY | CL | SY | CL |
| 5 | SY | CL | SY | CL | SY | CL | SY | CL | SY | CL |
| 6 | SY | CL | SY | CL | SY | CL | SY | CL | SY | CL |

Note:
“CL” indicates clear liquid; “SY” indicates slightly yellow.

| | | | | | | |
|---|---|---|---|---|---|---|
| Formula ID | 1 | 2 | 3 | 4 | 5 | 6 |

| T0 | | | ✓ | ✓ | ✓ | ✓ | ✓ | ✓ |
|---|---|---|---|---|---|---|---|---|
| | Shaking | 1 W | ✓ | ✓ | ✓ | ✓ | ✓ | ✓ |
| | F/T | 3 Cycles | ✓ | ✓ | ✓ | ✓ | ✓ | ✓ |
| | | 5 Cycles | ✓ | ✓ | ✓ | ✓ | ✓ | ✓ |
| | 40° C. | 1 W | ✓ | ✓ | ✓ | ✓ | ✓ | ✓ |
| | | 2 W | ✓ | ✓ | ✓ | ✓ | ✓ | ✓ |

Note:
✓: Essentially free of visible particle.

The results of SEC-HPLC purity (%) test:

| | | | | Shaking | | | F/T | | | 40° C. | | | | | |
|---|---|---|---|---|---|---|---|---|---|---|---|---|---|---|---|
| Formula | T0 | | | 1 W | | | 5 Cycles | | | 1 W | | | 2 W | | |
| ID | Mono | Aggr | Frac | Mono | Aggr | Frac | Mono | Aggr | Frac | Mono | Aggr | Frac | Mono | Aggr | Frac |
| 1 | 99.2 | 0.8 | 0 | 99.1 | 0.9 | 0 | 99.2 | 0.9 | 0 | 99.0 | 1.0 | 0 | 98.7 | 1.3 | 0 |
| 2 | 99.2 | 0.8 | 0 | 99.1 | 0.9 | 0 | 99.1 | 0.9 | 0 | 98.9 | 1.1 | 0 | 98.4 | 1.6 | 0 |
| 3 | 99.1 | 0.9 | 0 | 99.0 | 1.0 | 0 | 99.0 | 1.0 | 0 | 98.7 | 1.3 | 0 | 98.1 | 1.9 | 0 |
| 4 | 99.2 | 0.8 | 0 | 99.2 | 0.9 | 0 | 99.2 | 0.8 | 0 | 99.0 | 1.1 | 0 | 98.6 | 1.4 | 0 |
| 5 | 99.2 | 0.8 | 0 | 99.1 | 0.9 | 0 | 99.1 | 0.9 | 0 | 98.9 | 1.2 | 0 | 98.4 | 1.6 | 0 |
| 6 | 99.1 | 0.9 | 0 | 99.0 | 1.0 | 0 | 99.0 | 1.0 | 0 | 98.7 | 1.3 | 0 | 98.2 | 1.9 | 0 |

Note:
“Mono” indicates Monomer; “Aggr” indicates Aggregate; “Frac” indicates Fragment.

The results of non-reduced CE-SDS purity (%) test:

| | | | |
|---|---|---|---|
| Formula | Shaking | F/T | 40° C. |

| ID | T0 | 1 W | 5 Cycles | 1 W | 2 W |
|---|---|---|---|---|---|
| 1 | 97.9 | 97.3 | 97.9 | 97.4 | 96.3 |
| 2 | 98.2 | 97.0 | 98.2 | 97.5 | 97.0 |
| 3 | 97.9 | 97.2 | 98.0 | 97.6 | 97.2 |
| 4 | 97.9 | 96.9 | 98.1 | 97.4 | 96.4 |
| 5 | 98.1 | 97.1 | 97.7 | 97.6 | 97.7 |
| 6 | 97.4 | 97.1 | 97.3 | 96.8 | 97.6 |

### 5.5 Conclusion

The results of the visible particle inspection, SEC-HPLC purity detection and non-reduced CE-SDS purity detection show that AC102-6-1-1 has very good stability at a high protein concentration formulation, such as 62.2 mg/ml, 82.2 mg/ml and 100.4 mg/ml. Therefore, the AIAC can be used at a high concentration under the formulation of present disclosure.

Although specific embodiments of the disclosure have been described above, it is understood that they are for purposes of example only, and the scope of the disclosure is defined by the appended claims. Those skilled in the art can make various changes or modifications to the embodiments without departing from the spirit and scope of the disclosure, and such changes and modifications fall within the scope of the disclosure.

Sequencing List

SEQ ID No. 1: Ab0001-LCCT_{L}-HC Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGALPETGG
SEQ ID No. 2: Ab0001-LCCT_{L}-HC Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID No. 3: Ab0001-LC-HCCT Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID No. 4: Ab0001-LC-HCCT Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGKLPETGG
SEQ ID No. 5: Ab0001-LC-HCCT_{L} Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
SEQ ID No. 6: Ab0001-LC-HCCT_{L} Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGKGALPETGG
SEQ ID No. 7: Ab0001-LCCT-HC Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECLPETGG
SEQ ID No. 8: Ab0001-LCCT-HC Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID No. 9: Ab0001-LCCT-HCCT Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECLPETGG
SEQ ID No. 10: Ab0001-LCCT-HCCT Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGKLPETGG
SEQ ID No. 11: Ab0001-LCCT-HCCT_{L} Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECLPETGG
SEQ ID No. 12: Ab0001-LCCT-HCCT_{L} Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGKGALPETGG
SEQ ID No. 13: Ab0001-LCCTL-HCCT Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGALPETGG
SEQ ID No. 14: Ab0001-LCCTL-HCCT Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGKLPETGG
SEQ ID No. 15: Ab0001-LCCTL-HCCT_{L} Light chain:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGECGALPETGG
SEQ ID No. 16: Ab0001-LCCTL-HCCT_{L} Heavy chain:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKTH
TCPPCPAPELLGGPSVFLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGKGALPETGG
SEQ ID No. 17: LCDR1: RASQDVNTAVA
SEQ ID No. 18: LCDR2: SASFLYS
SEQ ID No. 19: LCDR3: QQHYTTPPT
SEQ ID No. 20: HCDR1: DTYIH
SEQ ID No. 21: HCDR2: RIYPTNGYTRYADSVKG
SEQ ID No. 22: HCDR3: WGGDGFYAMDY
SEQ ID No. 23: VL:
DIQMTQSPSSLSASVGDRVTITCRASQDVNTAVAWYQQKPGKAPKLLIYSASFLYSGVPSRFSGSRSGTDFTLTIS
SLQPEDFATYYCQQHYTTPPTFGQGTKVEIKRTV
SEQ ID No. 24: VH:
EVQLVESGGGLVQPGGSLRLSCAASGFNIKDTYIHWVRQAPGKGLEWVARIYPTNGYTRYADSVKGRFTISADT
SKNTAYLQMNSLRAEDTAVYYCSRWGGDGFYAMDYWGQGTLVTVSS

1. A pharmaceutical composition, comprising:
an antibody-immune agonist conjugate (AIAC),
a buffer, and
a stabilizer;
wherein the AIAC has the structure of formular (II-1), (II-2) or the mixture thereof: 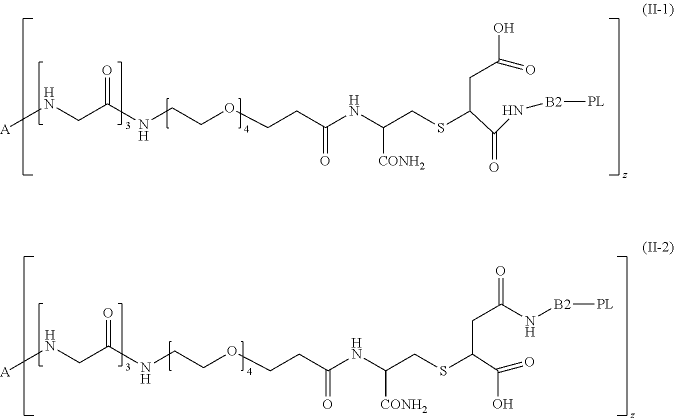
wherein B2 is —(CH₂)ₖ(CO)—NH—(C₂H₄—O)ⱼ— or —(CH₂)ₖC(O)—(NH—CR¹R²—C(O))_{d}—, k is an integer of 1 to 5, j is an integer of 1 to 3, d is an integer of 1 or 2, R¹ and R² are each independently selected from hydrogen, —OH, —NH₂, —C₁₋₆ alkyl;
PL is a payload which is linked to the B2 moiety,
preferably, PL is Resiquimod 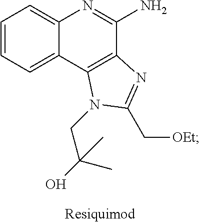 [SMILES: CCOCc1nc2c(N)nc3ccccc3c2n1CC(C)(C)O]
z is an integer of 1 to 4; preferably 1 to 2;
A is an antibody comprising a V_{L} and a V_{H},
wherein the V_{L} comprises LCDR1 having the amino acid sequence of SEQ ID NO: 17, LCDR2 having the amino acid sequence of SEQ ID NO: 18, and LCDR3 having the amino acid sequence of SEQ ID NO: 19,
wherein the V_{H} comprises HCDR1 having the amino acid sequence of SEQ ID NO: 20, HCDR2 having the amino acid sequence of SEQ ID NO: 21, and HCDR3 having the amino acid sequence of SEQ ID NO: 22;
wherein the antibody is modified by introduction of the ligase donor substrate recognition sequence.

2. The pharmaceutical composition of claim 1, wherein the antibody comprises a V_{L} having the amino acid sequence of SEQ ID NO: 23 and a V_{H} having the amino acid sequence of SEQ ID NO: 24.

3. A pharmaceutical composition, comprising:
an antibody-immune agonist conjugate (AIAC),
an acidic buffer, and
a stabilizer;
wherein the AIAC has the structure of formular (II-1), (II-2) or the mixture thereof: 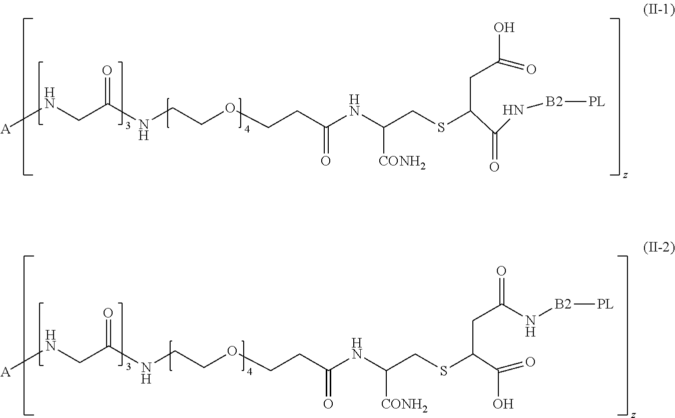
wherein B2 is —(CH₂)ₖ(CO)—NH—(C₂H₄—O))ⱼ— or —(CH₂)ₖC(O)—(NH—CR¹R²—C(O))_{d}—, k is an integer of 1 to 5, j is an integer of 1 to 3, d is an integer of 1 or 2, R¹ and R² are each independently selected from hydrogen, —OH, —NH₂, —C₁₋₆ alkyl;
PL is a payload which is linked to the B2 moiety,
preferably, PL is Resiquimod 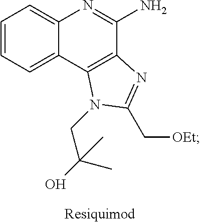 [SMILES: CCOCc1nc2c(N)nc3ccccc3c2n1CC(C)(C)O]
z is an integer of 1 to 4; preferably 1 to 2;
A is an antibody comprising
a light chain having the amino acid sequence of SEQ ID NO: 1 and a heavy chain having the amino acid sequence of SEQ ID NO: 2; or
a light chain having the amino acid sequence of SEQ ID NO: 3 and a heavy chain having the amino acid sequence of SEQ ID NO: 4; or
a light chain having the amino acid sequence of SEQ ID NO: 5 and a heavy chain having the amino acid sequence of SEQ ID NO: 6; or
a light chain having the amino acid sequence of SEQ ID NO: 7 and a heavy chain having the amino acid sequence of SEQ ID NO: 8; or
a light chain having the amino acid sequence of SEQ ID NO: 9 and a heavy chain having the amino acid sequence of SEQ ID NO: 10; or
a light chain having the amino acid sequence of SEQ ID NO: 11 and a heavy chain having the amino acid sequence of SEQ ID NO: 12; or
a light chain having the amino acid sequence of SEQ ID NO: 13 and a heavy chain having the amino acid sequence of SEQ ID NO: 14; or
a light chain having the amino acid sequence of SEQ ID NO: 15 and a heavy chain having the amino acid sequence of SEQ ID NO: 16.

4. The pharmaceutical composition of claim 1, wherein R¹ and R² are each independently hydrogen or C₁₋₆ alkyl; preferably, R¹ and R² are each independently both hydrogen or both C₁₋₆ alkyl; more preferably, R¹ and R² are both hydrogen.

5. The pharmaceutical composition of claim 4, wherein k is 2, and/or j is 1, and/or d is 1.

6. The pharmaceutical composition of claim 4, wherein the AIAC is selected from the following structures, or the mixture thereof: 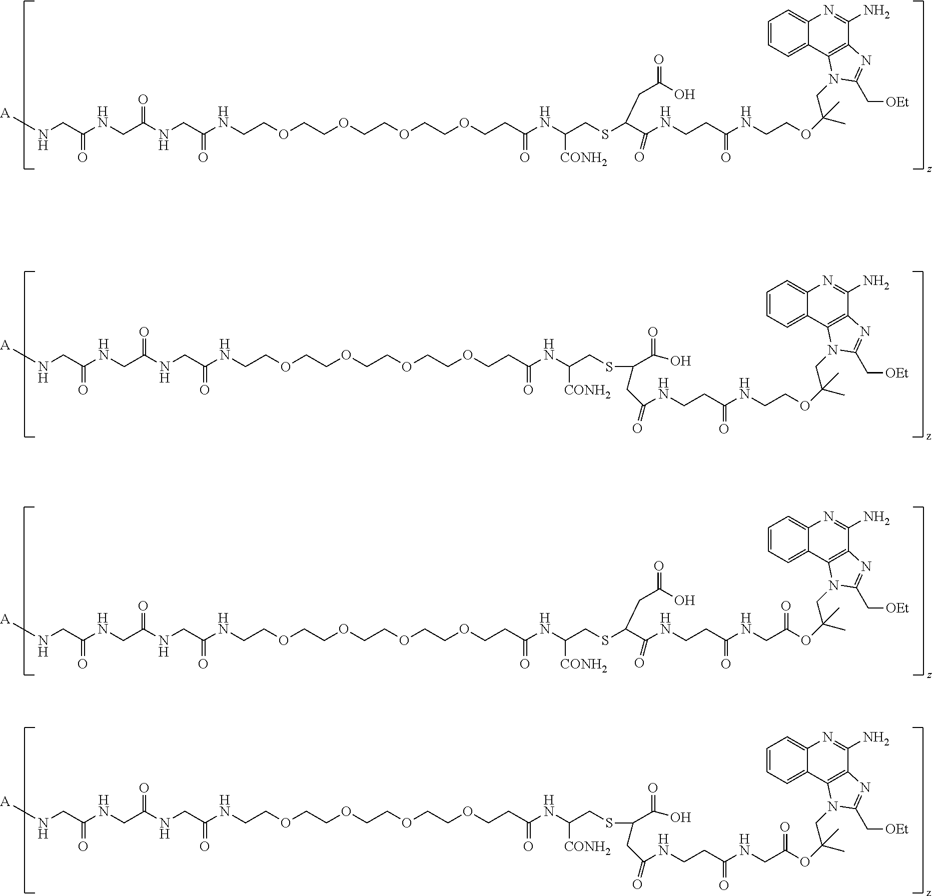
z is an integer of 1 to 4; preferably 1 to 2;
A is an antibody comprising a V_{L} and a V_{H}, wherein the V_{L} comprises LCDR1 having the amino acid sequence of SEQ ID NO: 17, LCDR2 having the amino acid sequence of SEQ ID NO: 18, and LCDR3 having the amino acid sequence of SEQ ID NO: 19, wherein the V_{H} comprises HCDR1 having the amino acid sequence of SEQ ID NO: 20, HCDR2 having the amino acid sequence of SEQ ID NO: 21, and HCDR3 having the amino acid sequence of SEQ ID NO: 22;
wherein the antibody is modified by introduction of the ligase donor substrate recognition sequence; or
A is an antibody comprises a light chain having the amino acid sequence of SEQ ID NO: 1 and a heavy chain having the amino acid sequence of SEQ ID NO: 2.

7. The pharmaceutical composition of claim 1,
wherein the conjugate of the AIAC has a drug to antibody ratio (DAR) of an integer or non-integer of 1 to 4, 1-4, 1-3.5, 1-3, 1-2.5, 1-2, 1.5-2, 1.6-2, or 1.7-2.

8. The pharmaceutical composition of claim 1, wherein
the acidic buffer comprises one or more of Sodium Acetate, Sodium Citrate and Histidine; preferably Histidine; or
the acidic buffer is selected from citrate buffer, phosphate buffer, histidine buffer and acetate buffer.

9. The pharmaceutical composition of claim 1, wherein the concentration of the buffer is 10-40 mM; or the concentration of the buffer is 15-25 mM; preferably 20 mM.

10. The pharmaceutical composition of claim 1, wherein the pH of pharmaceutical composition is 4.0-6.0; preferably the pH is 5.5.

11. The pharmaceutical composition of claim 1, wherein the stabilizer comprises one or more of sucrose, trehalose dihydrate, trehalose and sorbitol; preferably sucrose.

12. The pharmaceutical composition of claim 1, wherein the stabilizer comprises one or more of 5-15% (W/V) sucrose, trehalose dihydrate, trehalose and sorbitol; preferably 9% (W/V) sucrose, trehalose dihydrate, trehalose and sorbitol; more preferably 9% (W/V) sucrose.

13. The pharmaceutical composition of claim 1, wherein the pharmaceutical composition comprises surfactant.

14. The pharmaceutical composition of claim 13, wherein the surfactant comprises 0.05-0.5 mg/ml tween 20 or tween 80; preferably 0.2 mg/ml tween 20.

15. The pharmaceutical composition of claim 1, comprising:
10-40 mM histidine buffer, pH 4.0-6.0;
5-15% (W/V) sucrose;
0.05-0.5 mg/ml tween 20; or
15-25 mM histidine buffer, pH 4.0-6.0;
5-15% (W/V) sucrose;
0.05-0.5 mg/ml tween 20.

16. The pharmaceutical composition of claim 1, comprising:
20 mM histidine buffer, pH 5.5;
9% (W/V) sucrose;
0.2 mg/ml tween 20.

17. The pharmaceutical composition of claim 1, wherein the concentration of the protein of AIAC is 18 mg/ml-110 mg/ml; or the concentration of the protein of AIAC is 18 mg/ml-66 mg/ml.
